(19) United States
(12) Patent Application Publication
(10) Pub. No.: US 2024/0382587 A1
(43) Pub. Date: Nov. 21, 2024

(54) CELLULAR THERAPY TO SUPPRESS IMMUNE RESPONSE

(71) Applicants: THE TRUSTEES OF INDIANA UNIVERSITY, Bloomington, IN (US); UNIVERSITY OF FLORIDA RESEARCH FOUNDATION, INC., Gainesville, FL (US)

(72) Inventors: Moanaro BISWAS, Fishers, IN (US); Jyoti RANA, Indianapolis, IN (US); Todd Michael BRUSKO, Gainesville, FL (US)

(21) Appl. No.: 18/691,666

(22) PCT Filed: Oct. 6, 2022
(86) PCT No.: PCT/US2022/077696

§ 371 Date: Mar. 13, 2024

Related U.S. Application Data
(60) Provisional application No. 63/253,266, filed on Oct. 7, 2021.
(60) Provisional application No. 63/399,889, filed on Aug. 22, 2022.

(51) Int. Cl. A61K 39/00; A61P 37/06; C07K 14/705; C07K 14/715; C07K 16/36

(52) U.S. Cl. CPC A61K 39/4611; A61K 39/4621; A61K 39/4632; A61K 39/46434; A61P 37/06; C07K 14/70517; C07K 14/70521; C07K 14/715; C07K 16/36; A61K 2239/13; A61K 2239/17; C07K 2317/622

(57) ABSTRACT

Disclosed herein are compositions comprising engineered antigen-specific Tregs that suppress antibody formation against the soluble therapeutic protein factor VIII in an MIC-independent fashion. Complexing TCR-based signaling with single-chain variable fragment (scFv) recognition to generate TCR fusion construct (TRUC)-Tregs delivered controlled antigen-specific signaling via engagement of the entire TCR complex, thereby directing functional suppression of the FVIII-specific antibody response.

### CROSS REFERENCE TO RELATED APPLICATIONS

This application claims priority to the following: U.S. Provisional Patent Application No. 63/253,266 filed on Oct. 7, 2021 and U.S. Provisional Patent Application No. 63/399,889 filed on Aug. 22, 2022, the disclosures of which are expressly incorporated herein.

### GOVERNMENT LICENSE RIGHTS

This invention was made with government support under AI042288 and DK106191 awarded by National Institutes of Health. The Government has certain rights in the invention.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 13 kilobytes xml file named “29920-372450.xml,” created on Sep. 27, 2022.

### BACKGROUND

Anti-drug antibodies (ADAs) can develop in response to repeated administrations of biologics such as protein or enzyme replacement therapy, monoclonal antibodies, antibody drug conjugates, and immunotoxins. The formation of ADAs interferes with the effect of the drug or neutralizes it, thereby altering its pharmacokinetic (PK) and pharmacodynamic (PD) properties and reducing efficacy. Under certain circumstances, ADAs may also lead to severe adverse reactions such as hypersensitivity or even life-threatening anaphylaxis. Predicting which patients will develop ADA is difficult, and effective ADA mitigation strategies have yet to be developed.

While responses to biologics are characterized as “antibody mediated responses”, there is an essential role of CD4+ T cell help in supporting generation of class-switched ADAs through cognate interactions with antigen specific B cells and promoting memory and long-lived plasma cell development. CD4+ T cell help is, in turn, strongly dependent on modulation by Regulatory T cells (Tregs). Tregs are a specialized subpopulation of T cells that act to suppress immune response, thereby maintaining homeostasis and self-tolerance. Considering the indispensable role of Tregs in suppressing ADA formation, applicant has investigated the relevance of cellular engineered Treg therapy for inducing tolerance to biologics. Novel approaches to induce specific tolerance, especially during initial exposure, are expected to play significant roles in efforts to prevent or reduce unwanted ADA responses.

Tregs control immune responses in autoimmune disease, transplantation, and enable antigen-specific tolerance induction in protein replacement therapies. Tregs can exert a broad array of suppressive functions through their T cell receptor (TCR) in a tissue-directed and antigen-specific manner. As disclosed herein, this capacity can be harnessed for tolerance induction by “redirecting” polyclonal Tregs to overcome low inherent precursor frequencies and simultaneously augment suppressive functions.

FoxP3 expressing regulatory T cells (Tregs) are crucial drivers of central and peripheral tolerance and are therefore an ideal cellular therapeutic tool for antigen-specific tolerance induction. In human clinical trials, single infusions of polyclonal Tregs can successfully prevent or attenuate autoimmune disease as well as allogeneic hematopoietic cell or solid organ transplant rejection, thus reducing dependency on immunosuppressive drugs. Tregs can further be redirected for antigen specificity using cutting-edge cellular engineering mechanisms, thereby improving targeted suppression at lower effective doses.

One strategy to engineer specificity in polyclonal Tregs is to express a chimeric antigen receptor (CAR), which is a synthetic molecule that combines extracellular single chain variable fragments (scFv) of an antibody with primary TCR signaling and costimulatory moieties. Alternatively, this strategy can potentially be applied to FoxP3 engineered conventional T cells (Tconv) under conditions of Treg scarcity. CAR expression combines antigen specificity and cell signaling without the requirement for MHC class II restriction in a diverse patient group. CAR engineered Tconv have been shown to be highly effective at eradicating B cell leukemias that are resistant to standard therapies, whereas studies with CAR Tregs show promise in models of autoimmune disease and allograft rejection, with a first in man clinical trial soon to be launched for solid organ transplantation (phase I/II STEADFAST trial, TX200, Sangamo therapeutics).

To date, CAR Treg design has been modeled on CAR Tconv constructs for cancer by employing second-generation CD3ζ and costimulatory CD28 or 4-1BB signaling domains. Moreover, most CAR molecules have been designed to recognize membrane-bound surface antigens, with a major gap in understanding the mechanism of action for soluble antigens.

As an alternative to CAR Treg design, engineered TRUC Tregs can be provided that utilize “TCR-like” signaling where the endogenous TCR-CD3 signaling is reconfigured to respond to scFv-based recognition in Tregs. The TRUC design differs from the traditional CAR design in many respects. As opposed to 2nd and 3^{rd} generation CARs which encode for primary (CD3ζ) and costimulatory (CD28/4-1BB/ICOS) intracellular signaling domains in tandem, TRuCs are true hybrid BCR/TCR products. The TRUC receptor can incorporate the same scFv as a CAR into the Treg's endogenous TCR-CD3 complex by tethering the scFv to a CD3 subunit, in this case, CD3ε (FIG. 2A). Therefore, antigen specificity remains the same. TRUC Tregs adopt TCR internal signaling machinery by forming a ternary complex with endogenous TCR and CD3 chains. Incorporation of the TRUC construct into the TCR-CD3 complex limits surface receptor density and more faithfully mimics physiological TCR signaling, therefore delivering functional suppression of undesirable immune responses, such as the development of anti-drug antibodies (ADA).

Hemophilia A is an example of a disease where the formation of ADAs interfere with the efficacy of the standard treatment. Hemophilia A is an inherited x-linked bleeding disorder resulting from mutations causing a loss in functional factor VIII (FVIII) protein. Current treatment involves almost daily infusions of plasma derived or recombinant FVIII, with an aggregate lifetime cost of up to 25 million USD per patient. Patients with severe mutations develop antibodies (inhibitors) to the replacement clotting factor, which can neutralize treatment. Thus, hemophiliacs that develop inhibitors are at an increased risk for bleeds and require treatment with either expensive bypassing agents or immune tolerance induction (ITI) therapy, which requires daily infusion of high doses of coagulation factor that can last from months to years and is only effective in ˜60-70% of hemophilia A patients. Accordingly, there is a need for a therapeutic approach to counteract a patient's immune response to the administration of replacement clotting factor that will not result in off target immunosuppression or general immunosuppression.

As disclosed herein a cellular therapy using modified Tregs (TRUC Tregs) suppress the formation of antibodies against exogenous sources of protein factor VIII, and can act as preventive therapy, or as adjunct therapy with conventional immune tolerance induction (ITI) regimens.

### SUMMARY

In accordance with one embodiment the present disclosure is directed to therapeutic compositions and methods to prevent an inappropriate immune response, including an anti-drug antibodies (ADA) response to exogenous therapeutics administered to subjects to treat a particular disease or condition. More particularly, as disclosed herein engineered TRUC Tregs that utilize “TCR-like” signaling are provided where the endogenous TCR-CD3 signaling is reconfigured to respond to scFv-based recognition in Tregs. This synthetic construct is introduced into a regulatory T cell (optionally via retrovirally transduction), which provides the regulatory T cell with antigen recognizing properties. The original construct is called a TCR fusion construct (TRUC), wherein the variable heavy (VH) and light (VL) chains of an antibody with specificity to a therapeutic agent (or other inappropriate target of an immune response) are fused to a subunit of the T cell receptor called CD3 epsilon. These antigen specific regulatory T cells form a “living drug” or “cellular therapy”, where they suppress antibody formation to the therapeutic agent being administered to the patient.

In one embodiment modified Tregs (TRUC Tregs) are administered to patients that are receiving therapeutic exogenous proteins (or other therapeutic agents) to suppress the formation of antibodies against the therapeutic proteins/agents, wherein the variable heavy (VH) and light (VL) chains of an antibody with specificity to the therapeutic exogenous protein are fused to a subunit of the T cell receptor called CD3 epsilon. In one embodiment the modified Tregs (TRUC Tregs) are administered to patients that are receiving therapeutic exogenous proteins to supplement defective or insufficient endogenous protein production by the patient. In one embodiment infusions of autologous Tregs are used to prevent or attenuate an inappropriate immune response such as is found in autoimmune disease or transplant rejection.

In one embodiment the methods are directed to hemophilia patients that are receiving exogenous sources of protein factor VIII or other blood clotting factors. As disclosed herein antigen-specific Tregs can be engineered to suppress antibody formation against exogenous supplementation of proteins such as the administration of soluble therapeutic protein factor VIII, in an MHC-independent fashion. Surprisingly, high-affinity chimeric antigen receptor (CAR)-Treg engagement induced a robust effector phenotype that was distinct from the activation signature observed for endogenous thymic Tregs (t-Tregs), which resulted in the loss of suppressive activity. Complexing T cell receptor (TCR)-based signaling with single-chain variable fragment (scFv) recognition to generate a TCR fusion construct, (TRUC), when transfected in to Tregs (to form TRUC Tregs) and administered to a subject was found to deliver controlled antigen-specific signaling via engagement of the entire TCR complex, thereby directing functional suppression of the FVIII-specific antibody response.

TRUC combines antibody specificity for a target antigen (e.g., FVIII protein) with T cell signaling, wherein a TRUC molecule expressed on a regulatory T cell (i.e. a “TRUC Treg”) will recognize soluble exogenously administered FVIII and will signal release of immunosuppressive cytokines and other factors, which will suppress cell types that are involved in initiating an inhibitory antibody response to FVIII protein replacement therapy. FVIII TRUC Tregs are generated by retroviral transduction of polyclonal Tregs, which are then ex vivo expanded for 7-14 days. Finally FVIII TRUC Tregs are adoptively transferred into a patient (potentially hemophilia A patients) prior to, or simultaneously with, initiation of FVIII protein replacement therapy, or in patients (potentially hemophilia A patients) with established antibodies against FVIII protein, where cellular therapy with FVIII TRUC Tregs can potentially accelerate conventional immune tolerance induction and lead to more sustained tolerance to FVIII.

In one embodiment the TRUC molecule consists of a single chain variable fragment (scFv) of an antibody with specificity to FVIII, which is fused to the N-terminus of a murine TCR epsilon chain (see FIG. 2A). In one embodiment the scFv specifically binds to amino acid residues 2125-2332 of human FVIII. In one embodiment the TRUC molecule comprises the amino acid sequence of SEQ ID NO: 2, wherein amino acids 1-22 represent the CD3 Epsilon signal sequence, amino acids 23-131 represent the FVIII VL Chain, amino acids 132-146 represent the linker peptide, amino acids 147-263 represent the FVIII VH Chain, amino acids 264-278 represent a peptide linker and amino acids 279-445 represent the Murine CD3 epsilon peptide (see FIG. 2B). TRuCs are surface expressed as a component of the TCR-CD3 complex and recognition of soluble FVIII by the scFv will result in TCR activation and suppression of inhibitory antibody responses to FVIII protein replacement therapy. Tregs can be transfected using any known technique and in one embodiment the Tregs are transduced with TRUC by retroviral transduction. TRuCs recognize FVIII directly, without the need for MHCII based presentation by antigen presenting cells. Therefore, there is no requirement for MHC matching for cellular therapy with engineered TRUC Tregs.

In accordance with one embodiment a method of inhibiting an immune response against exogenously administered clotting factor VIII (FVIII) is provided. In one embodiment the method comprises administering to said patient a regulatory T cell (Treg) that expresses a TCR fusion construct comprising a single chain variable fragment (scFv) having specificity for clotting factor FVIII fused to the N-terminus of a CD3 ε subunit, optionally via a peptide linker. In one embodiment the scFV specifically binds to amino acid residues 2125-2332 of human FVIII, and optionally further comprises hinge regions selected from either CD28 or CD8 of either murine or human origin.

In accordance with one embodiment a co-inhibitory molecule called Programmed Death Ligand 1 (PDL1) is retrovirally transduced with the FVIII specific TRUC construct (See FIG. 2B). PDL1 on FVIII TRUC Treg interact with “Programmed Death 1 (PD1)”, which is very highly expressed on cells that are involved in making an immune response to FVIII in a specialized zone called the germinal center. The co-administration of PDL1 has been found to enhance the suppressive properties of the antigen specific regulatory T cell.

In one embodiment a nucleic acid is provided that encodes a TCR fusion construct (the TRUC molecule) having at least 90%, 95% or 99% sequence identity to SEQ ID NO: 2 or SEQ ID NO: 4. In one embodiment the TRUC molecule comprises an amino acid sequence having at least 90%, 95% or 99% sequence identity to SEQ ID NO: 2 or SEQ ID NO: 4.

In accordance with one embodiment a nucleic acid is provided that encodes chimeric antigen receptor (the TRUC molecule) having at least 90%, 95% or 99% sequence identity to SEQ ID NO: 1 or SEQ ID NO: 3. In one embodiment the nucleic acid has at least 95% sequence identity to SEQ ID NO: 1 or SEQ ID NO: 3. In one embodiment the chimeric antigen receptor (the TRUC molecule) is the human equivalent of the TRUC molecule of SEQ ID NO: 2 or SEQ ID NO: 4.

### BRIEF DESCRIPTION OF THE DRAWINGS

FIGS. 1A & 1B provide the nucleic acid (FIG. 1A; SEQ ID NO: 1) and amino acid (FIG. 1B; SEQ ID NO; 2) sequence of the FVIII TRUC construct. For FIGS. 1A and 1B, the CD3 Epsilon signal sequence is shown in lower case; FVIII VL chain is shown in upper case; Linker 1 sequence is italicized; the FVIII VH Chain is shown in underlined upper case; Linker 2 is italicized; and the Murine CD3 epsilon sequence is in bold.

FIGS. 1C and 1D provide the nucleic acid (FIG. 1C; SEQ ID NO: 3) amino acid (FIG. 1D; SEQ ID NO: 4) sequence of the FVIII TRUC-PDL1 construct. For FIGS. 1C and 1D, the CD3 Epsilon signal sequence is shown in lower case; FVIII VL Chain is shown in upper case; Linker 1 sequence is italicized; the FVIII VH Chain is shown in underlined upper case; Linker 2 is italicized; the Murine CD3 epsilon sequence is in bold; the P2A sequence is italicized and underlined; and the Murine PDL1 sequence is in bold and underlined.

FIGS. 2A-2D. The TCR-CD3 complex regulates TRUC surface expression. FIG. 2A is a schematic drawing showing the surface organization and schematic representation of the FVIII TRUC construct as present on the Treg surface. The variable light (VL) and heavy (VH) regions of the FVIII specific scFv, linker, extracellular, transmembrane, and intracellular signaling regions of murine CD3ε domains are indicated. FIG. 2B provides a schematic representation and surface organization of the FVIII TRUC construct co-expressed with the PDL1 gene, FIG. 2C: Representative density and histogram plot of TRUC transduced Tregs (indicated by mScarlet reporter protein) to show binding of 1 IU/mL FVIIIFc and detection with α-human IgG Fc conjugated to AF647. FIG. 2D: Comparison of surface scFv expression between CAR and TRUC Tregs at comparable transduction levels (indicated by mScarlet MFI) by α-human F(ab′) 2 binding and detection with α-goat AF647. Surface expression of FVIII TRUC is dependent on incorporation into the TCR-CD3 complex. Single or co-transduction of FVIII CAR or TRUC and TCRα/β into the TCRα/β deficient murine T cell line 5KCα⁻/β⁻ was conducted. Incubation with 11U FVIIIFc and detection of frequencies of mScarlet⁺ cells that bind FVIIIFc by aFc conjugated to AF647 indicated dependence of TRUC but not CAR surface expression and CD69 upregulation on co-transduction with TCRα/β (data not shown). Data points are averages±SEM. * P<0.05, ** P<0.01, *** P<0.001, **** P<0.001 by unpaired t-test for (FIG. 2D).

FIGS. 3A-3L. TRUC Tregs exhibit controlled signaling in vitro. FIGS. 3A-3E provide data in bar graphs showing upregulation of Treg associated activation markers-CD69 (FIG. 3A), Ki67 (FIG. 3B), CD28 (FIG. 3C), CTLA4 (FIG. 3D) and FoxP3 (FIG. 3E) by BDD-FVIII stimulated murine TRUC Tregs at 48 h in vitro (lane 1: control; lane 2: FVII; lane 3: FIX). Controls include unstimulated cells, or stimulation with an irrelevant protein, FIX.

FIGS. 3F-3H: Comparison of pAKT (S473; FIG. 3F), pERK FIG. 3G) or pS6 (FIG. 3H) at indicated times following stimulation with low dose (0.1 IU/mL), high dose (5 IU/mL) BDD-FVIII or TCR triggering with α-CD3/28 microbeads by flow cytometry for WT CAR (solid line), TRUC (dotted line) Tregs.

FIGS. 3I & 3J: Comparison of western blot analysis for pERK (FIG. 3I) and pS6 (FIG. 3J) at indicated times following stimulation with high dose (5 IU/mL) or low dose (0.1 IU/mL) BDD-FVIII. Densitometric analysis for pERK and pS6 for WT CAR Treg (solid line) or TRUC Treg (dotted line) are indicated.

FIG. 3K: Detection of IL-2, IL-10, IL-4, IL-17 and IFNγ cytokines from BDD-FVIII stimulated TRUC and CAR Treg cell supernatants at 48 h in vitro. The results show FVIII TRUC Tregs produce significantly lower levels of IL-2, IL-10, IL-4, IL-17 and IFNγ cytokines as compared to FVIII CAR Treg in response to FVIII stimulation in vitro.

FIG. 3L: Normalized in vitro suppression of CTV labeled FVIII TRuC Tconv proliferation when co-cultured with FVIII TRUC Tregs at the indicated Treg: Tconv ratios. Cells were stimulated with high dose (5 IU/mL) or low dose (0.1 IU/mL) BDD-FVIII or left unstimulated for 72 h in vitro. Percentage suppression calculated as [(mean proliferation Tconv−mean proliferation Treg+ Tconv)/(mean proliferation Tconv)]×100%. Data points are averages±SEM. * P<0.05, ** P<0.01, *** P<0.001, **** P<0.001 by 1-way ANOVA with Dunnett's comparisons for (FIGS. 3A-3E), multiple unpaired t-tests for (FIGS. 3F-3H), 2-way ANOVA test with Tukey's multiple comparison for (FIGS. 3I-3J) and (FIG. 3K), 2-way ANOVA with Sidak's multiple comparisons for (FIG. 3L).

FIGS. 4A-4K. TRUC Tregs maintain suppressive phenotype in vivo. FIG. 4A: Timeline for assessing in vivo prevention of inhibitor formation by TRUC Treg cellular therapy. 5×10⁵ TRUC Treg, 5×10⁵ or 1×10⁶ freshly isolated tTreg cells were adoptively transferred into BALB/c F8e16^{-/-} recipient mice (n=5-8/group). Mice received 8 weekly IV injections of 1.5 IU BDD-FVIII and plasma samples were analyzed after the 4th and 8th injection (FIG. 4B) for functional inhibitors by Bethesda assay (lane 1: control; lane 2: FVIII TRUC; lane 3: FVIII; lane 4: FVIII TRUC-PDL 1) or by α-FVIII IgG1 ELISA (see FIG. 4C). Control mice received only BDD-FVIII injections without Treg cell transfer. FIG. 4D: In vivo persistence of 1×10⁶ adoptively transferred FVIII CAR or TRUC Tregs following 3×/week BDD-FVIII (+FVIII) or mock (−FVIII) injections (n=3/group). Total number of adoptively transferred (mScarlet⁺ FoxP3^{GFP+}) engineered Tregs per 1×10⁶ splenic CD4⁺ T cells are indicated on days 3, 7 and 14 post adoptive transfer. FIG. 4E-4J: Effect of repeated in vitro BDD-FVIII stimulations on the phenotype of FVIII CAR or TRUC Tregs. Expression of PD1 (FIG. 4E), CD69 (FIG. 4F), LAP (FIG. 4G), CTLA-4 (FIG. 4H), FoxP3 (FIG. 4I), and frequencies of FoxP3+ cells (FIG. 4J) were estimated at each time point following 4 daily stimulations with BDD-FVIII (▪). Controls were either mock stimulated (●) or stimulated with an irrelevant antigen, FIX (▴). Solid lines represent CAR Tregs and dotted lines represent TRUC Tregs. FIG. 4K: In vivo Treg stability of 1×10⁶ FVIII CAR or TRUC Tregs assessed pre- and post-adoptive transfer in BALB/c F8e16^{-/-} recipient mice (n=6/group). One half of the recipient animals received 2 daily BDD-FVIII injections. mScarlet and FoxP3^{GFP+} co-expression following BDD-FVIII (+FVIII) or mock (−FVIII) injection were measured on day 3. The bar graph of FIG. 4K presents the results, with data points representing averages±SEM. * P<0.05, ** P<0.01, *** P<0.001, **** P<0.001 by 2-way ANOVA with Sidak's multiple comparisons for (FIG. 4B) and (FIG. 4C), 2-way ANOVA with Tukey's multiple comparisons for (FIG. 4D), (FIGS. 4E-4J) and (FIG. 4K).

FIG. 5: Structural differences: CARs combine scFv based receptor with synthetic signaling. TRuCs tether scFv recognition to a TCR subunit like CD3ε and incorporate into the endogenous TCR-CD3 complex.

FIGS. 6A-6D: Adoptive transfer of FVIII specific Tfh cells elevates functional ADAs (FIG. 6A) and anti-FVIII IgG1 titers (FIG. 6B) in response to 4 weekly FVIII injections in HA mice (n=7-8; lane 1: FVIII; lane 2: Tfh+FVIII). A single injection of PD-L1Fc (50 μg) reduces functional ADAs (FIG. 6C) and anti-FVIII IgG1 titers (FIG. 6C) by 3- and 2-fold respectively (n=5-6; lane 1: FVIII; lane 2 PDL1 Fc+FVIII).

FIG. 7: FVIII TRUC Tregs co-expressing CXCR5/PD-L1 were compared to TRUC Tregs that do not co-express CXCR5 or PD-L1. FIG. 7 shows the migration of FVIII TRUCCXCR5 Tregs towards a CXCL12 or CXCL13 gradient using a transwell assay (n=3), indicating specificity of CXCR5 for CXCL13.

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. As used herein an exogenous sequence in reference to a cell is a sequence that has been introduced into the cell from a source external to the cell.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviation of the symptoms associated with a specific disorder or condition and/or preventing or eliminating said symptoms.

As used herein an “effective” amount or a “therapeutically effective amount” of a drug/cell therapy refers to a nontoxic but enough of the drug/cell therapy to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein an amino acid “substitution” refers to the replacement of one amino acid residue by a different amino acid residue.

As used herein, the term “conservative amino acid substitution” is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine (hCys)
V. Large, aromatic residues:
   Phe, Tyr, Trp, acetyl phenylalanine, napthylalanine (Nal)

As used herein the term “patient” without further designation is intended to encompass any warm-blooded vertebrate domesticated animal (including for example, but not limited to livestock, horses, cats, dogs and other pets) and humans receiving a therapeutic treatment in the presence or absence of a physician's supervision.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

### Embodiments

Dysregulation of Treg signaling contributes to pathogenesis of many autoimmune conditions and highlights safety concerns in clinical Treg cell therapy. In accordance with one embodiment methods are provided to counteract autoimmune reactions or the production of anti-drug antibodies (ADAs) that develop in patients receiving therapeutic agents. More particularly, the present disclosure is directed to compositions and methods for administering modified Tregs (TRUC Tregs) to patients that are receiving therapeutic exogenous proteins (or other therapeutic agents) to suppress the formation of antibodies against the therapeutic proteins/agents. In this embodiment, the endogenous TCR-CD3 complex of Tregs are modified by tethering an scFv to a CD3 subunit (optionally, CD3ε), wherein the variable heavy (VH) and light (VL) chains of an antibody with specificity to the therapeutic exogenous protein are fused to the CD3 subunit of the T cell receptor.

In one embodiment a method is provided for modulating an inappropriate immune response (e.g., either an autoimmune reactions or an ADA response. The method comprises administering autologous Tregs that have been modified to express a TCR fusion construct that specifically binds the agent that is the target of the autoimmune reaction or an ADA response. In accordance with one embodiment the TCR fusion construct comprises the variable heavy (VH) and light (VL) chains of an antibody having specificity to the target of the autoimmune reaction or an ADA response, and the CD3 epsilon subunit of the T cell receptor, wherein the variable heavy (VH) and light (VL) chains are fused to the CD3 epsilon peptide, optionally via a peptide linker. In one embodiment the variable heavy (VH) and light (VL) chains are part of a single chain variable fragment (scFv) having specificity for the target agent, wherein the scFv is fused to the N-terminus of the CD3ε subunit, optionally via a peptide linker. In one embodiment the CD3ε subunit comprises an amino acid having at least 90%, 95% or 99% sequence identity to SEQ ID NO: 6. In one embodiment the CD3ε subunit comprises a humanized derivative of the murine peptide of SEQ ID NO: 6, where the sequence has been modified to more closely resemble the human equivalent peptide. In one embodiment the scFv is linked to the CD3ε subunit via a peptide linker of 2-16 amino acids selected from the group consisting of glycine, alanine and serine. In one embodiment the peptide linker comprises the sequence of SEQ ID NO: 5.

In one embodiment the modified Tregs (TRUC Tregs) are administered to patients that are receiving therapeutic exogenous proteins to supplement defective or insufficient endogenous protein production by the patient, such as clotting factor VIII. In one embodiment infusions of autologous Tregs are used to prevent or attenuate autoimmune disease or transplant rejection.

In accordance with one embodiment, a method of modifying an inappropriate immune response in a patient receiving repeated doses of a therapeutic exogenous agent is provided wherein the method comprises administering modified Tregs to said patient to suppress the formation of antibodies against the therapeutic agent. In one embodiment the modified Tregs comprise a T cell receptor fusion construct (TRUC), where the TRUC comprises variable heavy (VH) and light (VL) chains of an antibody with specificity to the therapeutic agent, and a T cell receptor CD3 epsilon subunit, wherein said variable heavy (VH) and light (VL) chains are fused to the CD3 epsilon subunit optionally via a peptide linker. In one embodiment the administered therapeutic exogenous agent is Factor VIII, and the variable heavy (VH) and light (VL) chains of the TRUC have specificity for Factor VIII. In one embodiment the method comprises administering a modified Treg, optionally wherein the Treg is recovered from the subject to be treated, wherein the modified Treg comprises a TCR fusion construct comprising a single chain variable fragment (scFv) having specificity for Factor VIII, and a CD3ε subunit, wherein the scFv is fused to the N-terminus of a CD3ε subunit, optionally via a peptide linker. In one embodiment the TRUC will comprise a CD3ε subunit having an amino acid sequence of at least 95% sequence identity to SEQ ID NO: 6. In one embodiment the method comprises administering a modified autologous Treg that comprises a TRUC having an amino acid sequence of at least 95% sequence identity to SEQ ID NO: 2 or SEQ ID NO: 4.

The TRUC constructs of the present disclosure comprise the variable heavy (VH) and light (VL) chains and a CD3 ε subunit, wherein each of these three components can optionally be linked to one another via a peptide linker to form a linear polypeptide. In accordance with one embodiment the peptide linker is a 2-16 amino acid sequence comprising amino acids selected from glycine, serine, alanine, and threonine. In one embodiment the peptide linker is a 2-16 amino acid sequence comprises of glycine and serine amino acids. In one embodiment the peptide linker comprises the sequence of GGGGSGGGGSGGGGS (SEQ ID NO: 5). In one embodiment an scFv that specifically binds to the therapeutic agent is linked to the extracellular domain of CD3ε via a 3× glycine-serine linker. In one embodiment a reporter (optionally the mScarlet reporter) is separated from the TRUC construct by an IRES element.

As disclosed herein, in one embodiment, redirecting Treg specificity to clotting factor VIII was accomplished using two different approaches: expression of CAR or TRUC. CAR signaling can overstimulate signaling molecules and lead to a pro-inflammatory profile in transduced Tregs. The magnitude of CAR signaling is dependent on scFv affinity, receptor density, antigen dose, choice of co-stimulatory molecule, and cytokine signals, among other factors. It is known that CAR scFv can bind to antigen with up to 1000-fold higher affinity than TCRs, although the effect of affinity on signaling and functionality are not well studied. It can be agreed that CAR signaling in both Tconv and Treg is more rapid and intense compared to TCR stimulation. This can result in over-activation and apoptosis of CAR Tconv cells, affecting in vivo persistence. Over-activation and potentially fatal cytokine release syndrome are important safety concerns following administration of CAR T cell therapies for cancer. Approaches for tapering the magnitude of CAR signaling such as introducing targeted mutations in CD3ζ ITAMs has been carried out previously, with some success, although this strategy has not been tested for CAR Tregs. Decreasing the number of ITAM pairs from three to two or even one in the CAR construct can increase selectivity and prevent off target effects by increasing the activation threshold. As disclosed herein, although Tregs are inherently more resistant to AICD, introducing the ITAM1⁻ mutation in CD3ζ (CD247) improved CAR Treg persistence in vivo. Interestingly, tTregs are reported to preferentially express the alternatively spliced θ isoform of CD247, which naturally lacks the ITAM3 domain, rather than the CD3ζ isoform.

The TCR repertoire in Tregs is mostly distinct from that of Tconv cells, with an increased tendency towards self-specificity. Signals elicited by the TCR are greatly dampened in Treg cells. This regulation is evident in both primary signal initiation and co-stimulatory signal potentiation, such that several signaling molecules like CD3ζ, SLP76, MAPK/ERK, AKT, or S6 and calcium flux are attenuated in Tregs. In particular Tregs are shown to be defective in AKT phosphorylation at Serine 473 (S473), thus displaying a reduced phosphorylation of AKT substrates. We confirmed a similar pattern of dampened signaling of these pathways upon TCR triggering of CAR transduced Tregs, which was distinct from CAR Tconv cell signaling. Conversely, CAR stimulation of these same cells resulted in increased phosphorylation of many of these signaling mediators, including strong phosphorylation at AKT S473, affirming that CAR signaling differs from endogenous Treg signaling at multiple signaling nodes. We do not yet know the effect that unrestricted CD28z signaling would have on inhibitory signaling motifs such as the inhibitory immunoreceptor tyrosine-based inhibitory motif (ITIM) or the immunoreceptor tyrosine-based switch motif (ITSM) commonly overexpressed in Tregs such as CTLA-4, PD-1, TIGIT. These motifs are responsible for the inhibition of TCR function and are thought to be critical for Treg immunosuppressive function. For this study, we did not test the 4-1BB co-receptor as its incorporation into the CAR construct has previously been shown to be detrimental to Treg function.

It has been reported that Tregs can augment proliferation of T cells under strong stimulatory conditions. We were able to confirm this, as mice that were infused with FVIII CAR Tregs developed high inhibitor titers. Mutating either the PI3K or LCK binding motifs in the CD28 signaling domain partially controlled the exacerbation of inhibitors in recipient mice. However, this was insufficient to confer suppressive activity, as recipient mice still developed inhibitors in response to BDD-FVIII injections.

CAR Treg stimulation in vitro was accompanied by significant production of IFNγ, TNFα, IL-10 and IL-4. Altered cytokine production by CAR Tregs has also been reported in murine models of graft versus host disease (GvHD), where IFNγ production by CAR Tregs as well as target cell lysis in a granzyme B dependent manner was observed. Given that IL-10 has been shown to block antigen-specific T cell cytokines such as PI3K/AKT induced IFNγ by inhibiting the CD28 signaling pathway, we demonstrated that constitutive expression of murine IL-10 in FVIII CAR Tregs was able to completely abrogate IFNγ production. However, IL-10 overexpressing FVIII CAR Tregs were unable to suppress the development of inhibitors in recipient mice and combining IL-10 overexpression with CD28-YMNM or PYAP mutations did not contribute to suppression. Since IL-10 is also reported to promote the germinal center response and IgG class switching, additional studies are needed to determine the effect of IL10 dose, constitutive vs inducible expression and localized vs systemic IL-10 delivery for optimizing tolerance to FVIII.

An important consideration for CAR Tregs specific to soluble antigens like FVIII is whether contact dependent mechanisms are essential for suppression, either via direct contact with antigen-bound B cell or dendritic cell, or by modulation of antigen presenting cell (APC) function via co-stimulatory molecule binding and/or trogocytosis. A recent report demonstrated transient suppressive activity of human CAR Tregs specific to the A2 domain of FVIII in a murine hemophilia A model, although the use of a xenogeneic system made it difficult to fully determine the extent of suppression. It is not known whether the affinity of the A2 CAR used in that study was significantly lower to that of the BO2C11 antibody used here, which has a very high affinity of 10⁻¹¹M⁻¹. One notable difference between the two studies is that in vitro suppression was enhanced by the presence of autologous PBMC, indicating a requirement for contact mediated suppression, most likely with APC. We believe that these two independent studies are not contradictory, but rather raise important questions about the role of scFv affinity and requirement for contact dependent mechanisms of suppression.

We tested an alternative approach to engineer antigen specific Tregs by tethering FVIII scFv to the CD3ε subunit of the TCR-CD3 complex (producing the TRUC molecule), which can overcome the limitations of destabilizing effects mediated by rapid and strong CAR signaling. We and others observed TRUC to be expressed on the cell surface as a component of the TCR-CD3 complex. In fact, incorporation of TRUC into the TCR-CD3 complex regulated receptor density on the transduced cell surface, likely contributing to modulation of signaling. It is also possible that the TRUC-TCR-CD3 complex is subject to internalization and re-expression following single or repeated exposure to antigen, which further protects the transduced cell from chronic activation or exhaustion. A related study targeting a CAR to the TRAC locus was shown to avert tonic signaling in a mouse model of acute lymphoblastic leukemia by a mechanism of CAR internalization and post-stimulation replenishment of cell-surface CAR expression. TRuCs employ the entire TCR complex to signal, whereas CARs utilize only the CD3ζ moiety of the complex with limited signaling capacity and lack intrinsic autoregulation, although recent studies indicate that CARs can interact with endogenous TCR molecules. FVIII TRUC Tregs were phenotypically stable and did not express cytolytic markers. Functionally, FVIII TRUC Tregs were immunosuppressive and prevented the formation of inhibitors to FVIII.

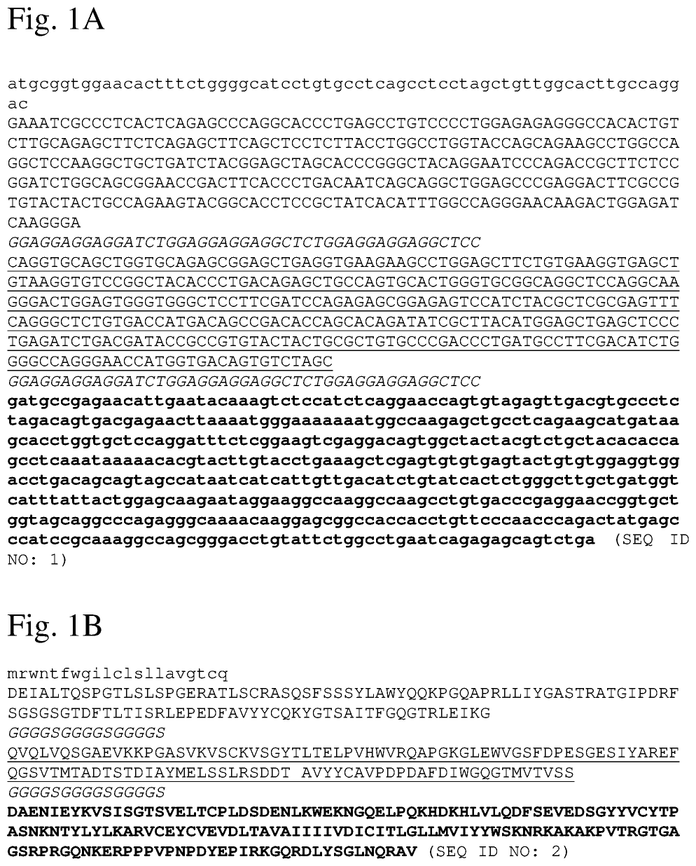

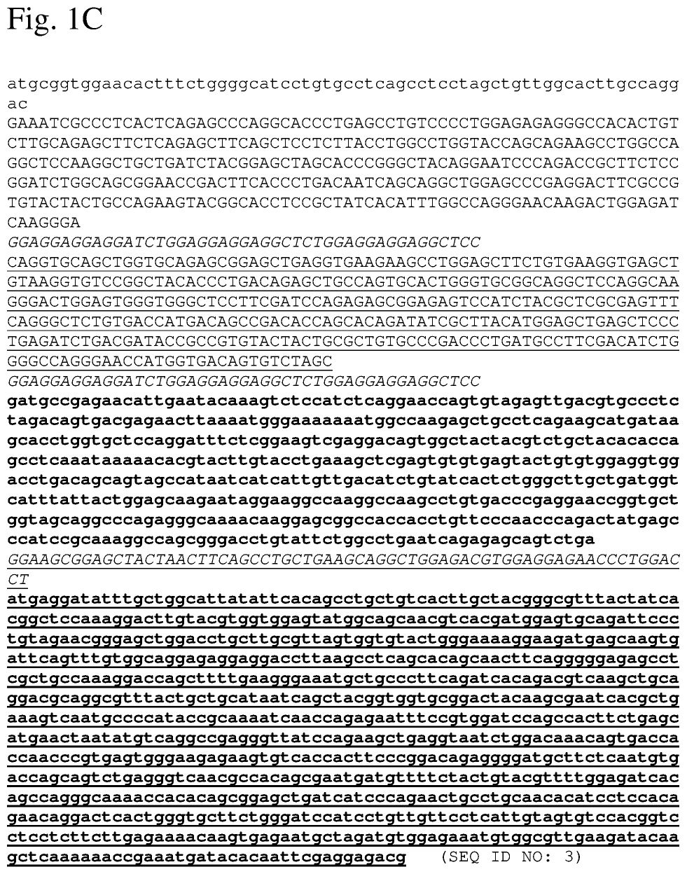

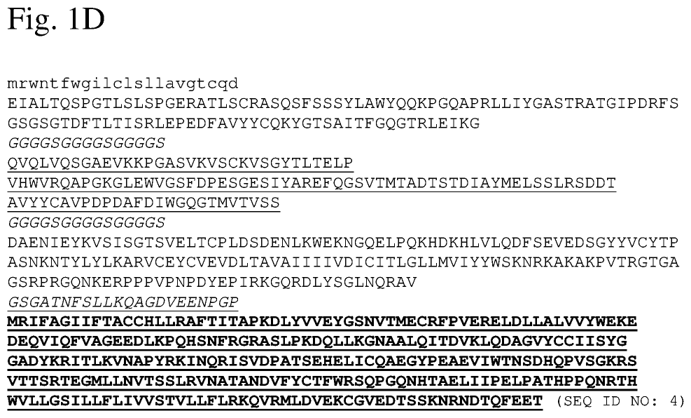

In accordance with one embodiment the addition of a co-inhibitory molecule called Programmed Death Ligand 1 (PDL1) has been found to enhance the suppressive properties of the antigen specific regulatory T cell that is retrovirally transduced with the FVIII specific TRUC construct. The resulting FVIII PDL1 TRUC construct comprises the variable heavy and light chains of an antibody that recognizes FVIII, derived from a hemophilia A patient, that is fused to the CD3 epsilon chain via a linker. A porcine teschovirus (P2A cleavage peptide) separates the FVIII TRUC DNA from the encoded PDL1 chain, so that they are expressed from a single transcript but subsequently cleaved into 2 different proteins. This entire DNA construct is retrovirally transduced into a regulatory T cell so that the FVIII PDL1-TRUC Treg expresses both the FVIII TRUC and PDL1. The FVIII-TRUC-PDL1 construct is superior in suppressing antibody responses to FVIII as compared to the parent FVI 11-TRUC construct (See sequences of FIGS. 1A-1D).

### Enhancing Treg Stability

In accordance with one embodiment methods are provided to enhance engineered Treg stability, localization, persistence, and suppressive properties in vivo. We propose to co-express the chemokine receptor CXCR5 with the co-inhibitory molecule programmed death ligand-1 (PDL1), presenting an innovative strategy to integrate antigen specific suppression at the site of ADA development with co-inhibitory “back-signaling” to increase Treg stability and suppressive capacity. This approach will minimize off-target suppressive effects, while maximizing localized, antigen specific suppression.

The combination of pharmacological immune modulation approaches with TRUC Treg cellular therapy presents an innovative alternative ITI approach to eradicate established ADAs, given that conventional ITI treatment has been used since late 1970, with unpredictable outcomes. This will also identify immunotherapeutic drugs that are compatible with engineered Treg therapy. These treatment combinations can also be extended to a number of disease indications with a breakdown in T and B cell tolerance, such as systemic lupus erythematosus or rheumatoid arthritis.

ADA formation in hemophilia is driven by CD4+ T follicular helper (Tfh) cells, which highly express the chemokine receptor CXCR5 and the co-inhibitory receptor PD1. Tfh cells activate cognate B cells to mature and proliferate within germinal centers (GCs) into high-affinity, class switched, ADA secreting plasma or memory B cells. Inhibitory signals delivered specifically to Tfh cells are anticipated to limit their activation and consequently, their ability to drive effective ADA responses. Since Tfh cells expressing the CXCR5 chemokine receptor are recruited to the B cell follicle or germinal center by chemotaxis along a CXCL13 gradient, we propose inducing CXCR5 co-expression in FVIII TRUC Treg to localize into B cell follicles/germinal centers, and PDL1 co-expression in FVIII TRUC Treg to inactivate PD-1 expressing Tfh cells and impair GC B cell responses. The PDL1 co-inhibitory receptor can bind PD-1, mediating immune suppression by a bifunctional mechanism of anergizing PD1 expressing T cells in a trans-fashion while reinforcing a self-suppressive phenotype by negative “back-signaling” in the PD-L1 expressing Treg.

Combined, CXCR5+PD-L1 co-expression is anticipated to enhance engineered Treg localization and suppressive capacity.

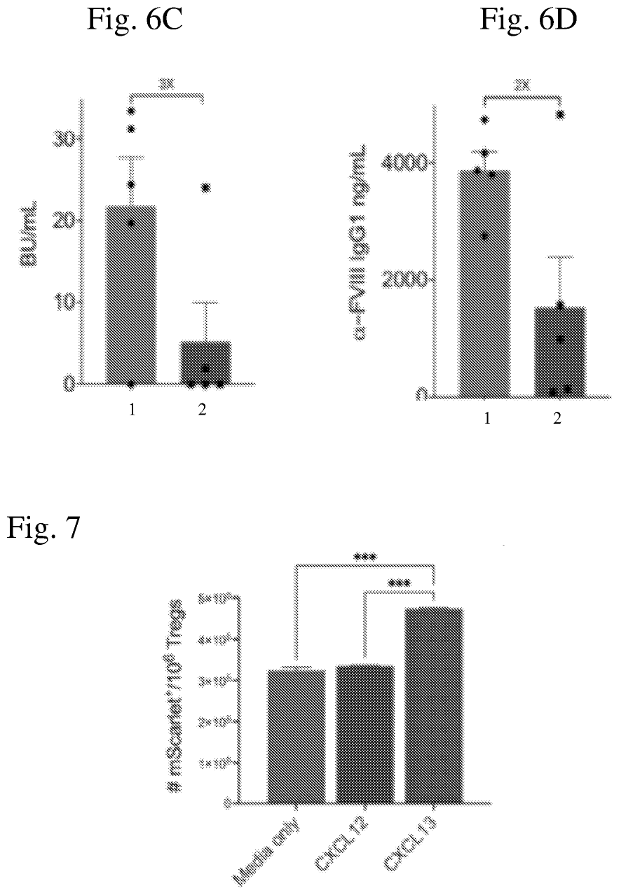

Our unpublished observations confirm a critical role for Tfh cells in ADA development. Adoptive transfer of 0.3×10⁶ Tfh cells from FVIII immunized mice (FVIII+Sigma adjuvant system, 2×/month) increased ADA responses by twofold in recipient HA mice (FIGS. 6A-6D). We further found that a single intraperitoneal injection of non-lytic mouse PDL1Fc (100 μg/mouse, Adipogen) followed 1 day later with FVIII weekly injections (1.5 IU/week for 4 weeks) caused HA mice to develop 3-fold lower ADA titers (5.16±4.7 BU/mL) compared to mock treated animals (18.15±6 BU/mL).

Collectively, these experiments demonstrate a critical role for Tfh cells in driving the ADA response to FVIII protein replacement therapy and a role for PDL1 in ADA tolerance.

As proof of concept, we generated FVIII (BO2C11) TRUC constructs co-expressing: i) CXCR5, and ii) *PDL1. We confirmed overexpression of CXCR5 or PDL1 in transduced Tregs by flow cytometric analysis (FIG. 7). Using an in vitro migration assay, we demonstrated that CXCR5 co-expressing TRUC Tregs migrated through a semi-permeable membrane in response to a CXCL13 gradient but not to CXCL12 further confirming functionality of these constructs (FIG. 7).

In accordance with one embodiment CXCR5 and PD-L1 expression will be combined in FVIII (BO2C11) TRUC Tregs. The scFv can be selected based on the desired target. In one embodiment we will use an scFv directed against FVIII (BO2C11). TRUC Tregs suppressed FVIII ADA formation for up to 8 weeks following adoptive transfer, but were not suppressive long term (12-16 week). We hypothesize that improving localization and suppressive back-signaling will enhance long-term suppression.

In accordance with embodiment 1, a method of modifying an inappropriate immune response in a patient receiving repeated doses of a therapeutic exogenous agent s provided wherein the method comprises
administering modified Tregs to said patient to suppress the formation of antibodies against the therapeutic agent, wherein said modified Tregs comprise a T cell receptor fusion construct (TRUC), and the TRUC comprises variable heavy (VH) and light (VL) chains of an antibody with specificity to said therapeutic agent; and a T cell receptor CD3 epsilon subunit, wherein said variable heavy (VH) and light (VL) chains are fused to the CD3 epsilon subunit optionally via a peptide linker. In accordance with one embodiment the peptide linker is a 2-16 amino acid sequence comprising amino acids selected from glycine, serine, alanine, and theronine. In one embodiment the peptide linker is a 2-16 amino acid sequence comprises of glycine and serine amino acids. In one embodiment the peptide linker comprises the sequence of GGGGSGGGGSGGGGS (SEQ ID NO: 5). In one embodiment an scFv that specifically binds to the therapeutic agent is linked to the extracellular domain of CD3ε via a 3× glycine-serine linker. In one embodiment a reporter (optionally the mScarlet reporter) is separated from the TRUC construct by an IRES element.

In accordance with embodiment 2 a method according to embodiment 1 is provided wherein said TRUC comprises a single chain variable fragment (scFv) having specificity for said therapeutic agent fused to the N-terminus of a CD3ε subunit, optionally via a peptide linker.

In accordance with embodiment 3 a method according to embodiment 1 or 2 is provided wherein the therapeutic agent is a protein.

In accordance with embodiment 4 a method according to any one of embodiments 1-3 is provided wherein the administered Tregs are autologous that have been modified to comprises said TCR.

In accordance with embodiment 5 a method according to any one of embodiments 1-4 is provided wherein the therapeutic agent is clotting factor FVIII.

In accordance with embodiment 6 a method according to any one of embodiments 1-5 is provided wherein the TCR fusion construct further comprises hinge regions selected from either murine or human CD28 or CD8.

In accordance with embodiment 7 a method according to any one of embodiments 1-6 is provided wherein said Treg further expresses a gene encoding chemokine receptor CXCR5.

In accordance with embodiment 8 a method according to any one of embodiments 1-7 is provided wherein said scFV specifically binds to amino acid residues 2125-2332 of human FVIII.

In accordance with embodiment 9 a method according to any one of embodiments 1-8 is provided wherein said TCR fusion construct comprises an amino acid sequence having at least 95% sequence identity to SEQ ID NO: 4.

In accordance with embodiment 10 a method of modifying an immune response in a patient administered clotting factor FVIII is provided, said method comprising administering to said patient a regulatory T cell (Treg) that expresses
i) a TCR fusion construct comprising
   a single chain variable fragment (scFv) having specificity for clotting factor FVIII fused to the N-terminus of a CD3ε subunit, optionally via a peptide linker; and
ii) an additional gene encoding Programmed Death Ligand 1 (PDL1).

In accordance with embodiment 11 a method according to embodiment 10 is provided wherein said Treg further expresses a gene encoding chemokine receptor CXCR5.

In accordance with embodiment 12 a method according to any one of embodiments 10-11 is provided wherein said scFV specifically binds to amino acid residues 2125-2332 of human FVIII.

In accordance with embodiment 13 a method according to any one of embodiments 10-12 is provided wherein the TCR fusion construct further comprises hinge regions selected from either murine or human CD28 or CD8.

In accordance with embodiment 14 a method according to any one of embodiments 10-13 is provided wherein said TCR fusion construct comprises an amino acid sequence having at least 95% sequence identity to SEQ ID NO: 4.

In accordance with embodiment 15 a method according to any one of embodiments 10-14 is provided wherein said TCR fusion construct comprises an amino acid sequence corresponding to SEQ ID NO: 4, wherein the murine amino acid sequences have been substituted with the corresponding human equivalent amino acid sequences.

In accordance with embodiment 16 a TCR fusion construct is provided that comprises a single chain variable fragment (scFv) having specificity for clotting factor FVIII fused to the N-terminus of a CD3ε subunit, optionally via a peptide linker.

In accordance with embodiment 17 a composition according to embodiment 16 is provided wherein the chimeric antigen comprises an amino acid sequence having at least 95% sequence identity to a sequence selected from SEQ ID NO: 2 or 4.

In accordance with embodiment 18 a nucleic acid construct is provided comprising a polynucleotide encoding a TCR fusion construct comprising single chain variable fragment (scFv) having specificity for clotting factor FVIII fused to the N-terminus of a CD3ε subunit, optionally via a peptide linker.

In accordance with embodiment 19 a construct according to claim 18 is provided wherein the construct further comprises a nucleic acid sequence encoding Programmed Death Ligand 1 (PDL1).

In accordance with embodiment 20 a construct according to claim 18 or 19 is provided wherein said nucleic acid has at least 95% sequence identity to a sequence selected from SEQ ID NO: 1 or 3.

In accordance with embodiment 21 an engineered Treg is provided, where the endogenous TCR-CD3 signaling of the Treg is reconfigured to respond to scFv-based recognition, said Treg expressing
i) a TCR fusion construct comprising
   a single chain variable fragment (scFv) having specificity for a preselected therapeutic agent, said scFv being fused to the N-terminus of a CD3ε subunit, optionally via a peptide linker.

In accordance with embodiment 22 an engineered Treg of embodiment 21 is provided wherein the Treg further expresses an additional gene, “Programmed Death Ligand 1 (PDL1)”.

In accordance with embodiment 23 an engineered Treg of embodiment 21 or 22 is provided wherein said Treg further expresses a gene encoding chemokine receptor CXCR5.

In accordance with embodiment 24 an engineered Treg of any one of embodiments 21-23 is provided wherein said scFv has specificity for clotting factor FVIII.

In accordance with embodiment 25 an engineered Treg of any one of embodiments 21-24 is provided wherein said scFV specifically binds to amino acid residues 2125-2332 of human FVIII.

In accordance with embodiment 26 an engineered Treg of any one of embodiments 21-25 is provided wherein the TCR fusion construct further comprises hinge regions selected from either murine or human CD28 or CD8.

In accordance with embodiment 27 an engineered Treg of any one of embodiments 21-26 is provided wherein said TCR fusion construct comprises an amino acid sequence having at least 95% sequence identity to SEQ ID NO: 4.

In accordance with embodiment 28 an engineered Treg of any one of embodiments 21-27 is provided wherein said TCR fusion construct comprises an amino acid sequence corresponding to SEQ ID NO: 4, wherein the murine amino acid sequences have been substituted with the corresponding human equivalent amino acid sequences.

### Example 1

### FoxP3 Engineered T Cells

In this study, we used a high-affinity CAR specific for coagulation factor VIII (FVIII) to suppress inhibitory antibody responses to replacement FVIII therapy in a murine model of hemophilia A. Mutations in the F8 gene can lead to reduced, misfolded, or complete lack of expression of FVIII in the blood, with severity of the condition consistent with the degree of residual clotting activity. Inhibitory antibodies to exogenously infused FVIII can neutralize the therapeutic protein in up to 30% of severe hemophilia A patients, thus interfering with treatment. A growing body of evidence suggests that immunomodulation by Tregs could offer a new treatment strategy in hemophilia A. We have previously shown that cellular therapy with either ex vivo expanded polyclonal Tregs, or FoxP3-transduced Tconv (i.e., de novo Tregs) enriched for antigen specificity are tolerogenic in a murine model of severe hemophilia A.

Here we sought to understand the effect of high-affinity CAR signaling on Treg stability, cytokine production, in vivo persistence and suppressive capacity. We analyzed the contribution of proximal and distal CD3 ζ immune receptor tyrosine-based activation motifs (ITAMs), as well as CD28 signaling motifs. We explored the alternative TCR fusion construct (TRUC), which was synthesized by fusing the FVIII scFv to the N-terminus of the TCRε subunit (Baeuerle et al, (2019) Nat Commun 10:2087).

We have confirmed that incorporation of the TRUC construct into the TCR-CD3 complex limited surface receptor density and more faithfully mimicked physiological T cell receptor (TCR) signaling. In vivo, TRUC Tregs suppressed the development of adaptive immune responses to FVIII. Complexing TCR based signaling with scFv recognition has not been tested earlier in Tregs and has the potential to engage not just the complete TCR machinery in an MHC unrestricted manner, but can also subject the cell to negative feedback mechanisms that are rapidly induced by TCR engagement to regulate signal output in response to antigen.

### Materials and Methods

### Mice

BALB/c Foxp3^{IRES-GFP} (Foxp3^{GFP}) mice were purchased from The Jackson Laboratory (Bar Harbor, ME). Hemophilia A mice with a deletion in exon 16 of the F8 gene (BALB/c F8e16^{-/-}) were originally provided by Dr. David Lillicrap (Queens University, Ontario, Canada). Animals were housed under specific pathogen-free conditions at Indiana University, Indianapolis, and treated under Institutional Animal Care and Use Committee-approved protocols. Both male and female mice were used as Treg donors for in vitro studies. Male mice were used for studies involving adoptive transfer or inhibitor formation.

### CAR and TRUC Constructs

The FVIII scFv was derived from an Epstein-Barr virus (EBV) transformed B-cell clone obtained from a hemophilia A patient (originally developed by Jacquemin and colleagues (Jacquemin, et al. (1998) Blood 92:496-506), kindly provided to us by Dr. David Scott, Uniformed Services University, MD). This B-cell clone (BO2C11) produces IgG4 directed against amino acid residues 2125-2332 of human FVIII, which corresponds to the carboxyl-terminus of C1 (residues 2125-2172) and the C2 (residues 2173-2332) light chain domains. The scFv was constructed from the V_{H} and V_{L} sequences (Creative Biolabs, Shirley, NJ). and fused to second-generation murine 28z CAR signaling sequences (kind gift from Dr. Angelica Loskog, Uppsala University, Sweden). Hinge regions from either murine CD28 or CD8 were incorporated with no observed differences in signaling. A Myc tag was cloned into the original construct (Genscript, Piscataway, NJ). Single amino acid mutations in ITAMs 1, 3, or 1+3 of CD3ζ or in the CD28 signaling domains were introduced by site directed mutagenesis (Genscript, Piscataway, NJ). Murine IL-10 was cloned downstream of the CAR sequence, separated by a P2A cleavage sequence (Genscript, Piscataway, NJ). FVIII specific TRUC was generated by complexing the BO2C11 FVIII scFv sequence to the N-terminus of murine CD3ε by a flexible linker (G4S)X3 (Genscript, Piscataway, NJ).

### Retroviral Transduction

FVIII TRUC sequences were inserted into the pMYs-IRES-mScarlet retroviral backbone. Transfer plasmids were transfected into the PlatE ecotropic retroviral packaging cell line (Cell Biolabs Inc, San Diego, CA) using either Viafect (Promega, Madison, WI) or polyethylenimine (PEI) transfection reagents, and supernatants were collected after 48 h. CD4⁺CD25⁻ Tconv or CD4⁺CD25⁺ Treg cells from BALB/c Foxp3^{GFP} mice were magnetically enriched using a mouse CD4⁺CD25⁺ Treg isolation kit (Miltenyi Biotec, Auburn, CA), further purified by cell sorting (FACS Aria II or FACS Aria SORP, BD Biosciences) and pre-activated for 48 h with a 1:1 bead to cell ratio using anti-CD3/28 mouse microbeads (Dynabeads, Invitrogen). High purity was ensured by “four-way purity” sort followed by post-sort flow analysis (99.5±0.3%). Tregs were cultured in Biotarget serum free media (Biological Industries, Cromwell, CT) supplemented with 5% fetal bovine serum (Atlanta Biologicals, Norcross, GA), 10,000 IU/mL penicillin, 10 mg/mL streptomycin, 1× GlutaMAX-1, 1 mmol/l sodium pyruvate, 10 mmol/l HEPES, 1× nonessential amino acids and 10 μmol/1 2-mercaptoethanol. Clinical grade recombinant hIL-2 (Proleukin/aldesleukin, Prometheus Therapeutics and Diagnostics, San Diego, CA) was added at a final concentration of 1000 IU/mL. Cells were transduced by spinoculation with retrovirus containing supernatants at 1200 xg for 90 minutes in non-tissue culture treated 6-well plates coated with 20 μg/mL retronectin (TakaraBio, Middleton, WI). Transduced cells were further purified by sorting for FoxP3^{GFP+}mScarlet⁺ cells and ex vivo expanded for 3-4 days in the presence of anti-CD3/28 microbeads at a 1:1 bead to cell ratio. 100 nM rapamycin (LC laboratories, Woburn, MA) was added under some conditions. Cells were rested for 4-6 h prior to functional in vitro or in vivo experiments

### Flow Cytometry

1×10⁶ FVIII CAR or TRUC transduced Tconv or Treg cells were plated in 12-well plates in Biotarget medium with 5% FBS without IL-2, before stimulation with 0.1, 1 or 5 IU/mL of recombinant human B domain deleted (BDD)-FVIII (Xyntha; Pfizer, New York, NY), FVIIIFc (Bioverativ, Cambridge, MA), FIXFc (Sanofi Genzyme, Cambridge, MA) and anti-human Fc (5 mg/mL, Biolegend, San Diego, CA) or anti-CD3/28 microbeads (1:1 bead to cell ratio). At 24-96 h, cells were first Fc-blocked with anti-CD16/32, then stained using antibodies against CD4 (BV421), GITR (BV510), RORγt (BV421) from BD Biosciences (San Jose, CA); CD69 (eFluor450), FoxP3 (eFluor660), GATA3 (PE/Cy7), Granzyme B (PerCP-eFluor 710) from eBioscience (San Diego, CA); CD4 (BV421), CTLA-4 (BV421), PD1 (BV605), CD28 (PerCP/Cy5.5), LAP (PE), Ki67 (PE/Cy7), CD69 (PE/Cy7), CD49b (APC/Cy7), GFP (A488), IRF4 (PE), T-Bet (BV605), anti-human IgG Fc (purified and AF647 conjugated), IL-10 (BV421), IL-4 (BV711), IL-17 (AF647), IFNγ (AF700), CD107a (BV711) from Biolegend (San Diego, CA); Myc (PE) from R&D Systems (Minneapolis, MN). CD107a staining was performed. Goat anti-human F(ab′) 2 antibody (Invitrogen) and anti-goat AF647 (Jackson Immunoresearch, West Grove, PA) were used for scFv surface detection. To analyze transcription factor expression, cells were first fixed with 2% paraformaldehyde and permeabilized using the Foxp3/Transcription Factor Staining Buffer (eBioscience, San Diego, CA). Data were analyzed using FCS Express v7 (DeNovo Software, Los Angeles, CA).

### Proliferation

For in vitro proliferation assay, cells were labeled with 3-5 μmol/l CTV (Invitrogen, Carlsbad, CA) prior to stimulation with BDD-FVIII or an irrelevant antigen (FIX, Benefix, Pfizer, New York, NY) and incubated for 72 h at 37° C. CTV dilution in proliferating relative to unstimulated Tregs was quantified via proliferation analysis in FCS Express v7. For in vivo proliferation and persistence, WT, ITAM1⁻, ITAM3⁻ FVIII CAR Tregs or FVIII TRUC Tregs were purified by FACS sorting and labeled with 3-5 μmol/l CTV. 1×10⁶ Tregs were adoptively transferred into recipient BALB/c F8e16^{-/-} mice (n=4/group), and one day later, mice were IV injected with 1.5 IU BDD-FVIII or left untreated. Mice were euthanized on days 3, 7/8 and 14 following adoptive transfer. Spleen CD4⁺ T cells were magnetically enriched and CTV⁺ FoxP3^{GFP+} mScarlet⁺ cells were quantified on a BD LSR Fortessa.

### In Vitro Suppression

For in vitro suppression, WT FVIII CAR or FVIII TRUC Tregs were purified by sorting, and incubated with 3-5 μmol/l CTV labeled FVIII CAR Tconv or FVIII TRUC Tconv responder cells respectively at varying ratios of Tregs:Tconv, while keeping Tconv numbers constant. Cells were stimulated with either high dose (5 IU/mL) or low dose (0.1 IU/mL) BDD-FVIII and acquired on a BD LSR Fortessa after 72 h at 37° C. Dilution of CTV in proliferating CAR Tconv or TRUC Tconv cells was quantified relative to unstimulated cells using proliferation analysis in FCS Express v7.

### Cytokine Detection

For intracellular cytokine staining, FVIII CAR or TRUC transduced cells were plated in 12 well plates in Biotarget medium with 5% FBS without IL-2, before stimulation with 5 IU/mL of BDD-FVIII, FIX or anti-CD3/28 microbeads (1:1 bead to cell ratio). Following 20-32 h of stimulation, Brefeldin A (3 μg/mL, eBioscience, San Diego, CA) was added for an additional 4 h. Cells were fixed and permeabilized with Cyto-Fast Fix/Perm buffer (Biolegend), and intracellular cytokine staining was performed for flow cytometry analysis. Additionally, supernatants were collected from stimulated cells at 48 h, and levels of IL-2, IL-4, IL-10, IL-35, IL-17, IL-21, and IFNγ were quantified by DuoSet ELISA kits according to manufacturer recommendations (R&D Systems, Minneapolis, MN).

### Phospho Flow

1×10⁶ FVIII CAR or TRUC Tconv or Tregs/well were plated in a 12 well plate in Biotarget medium without FBS or IL-2. Cells were stimulated with BDD-FVIII or anti-CD3/28 microbeads for 0, 10, 30 and 60 minutes, following which cells were immediately fixed with 2% paraformaldehyde. Fixed cells were permeabilized with 90% methanol for 30 minutes followed by staining for pERK (APC), pS6 (PacBlue), pAKT (S473, PE, APC) and pAKT (T308, APC), (Cell Signaling Technology, Danvers, MA) and analyzed by flow cytometry on a BD LSR Fortessa.

### Western Blot

CAR or TRUC Tregs were stimulated and fixed as described in Phospho flow. Fixed cells were lysed in ice-cold RIPA buffer containing protease and phosphatase inhibitors (Cell Signaling Technology, Danvers, MA). PAGE separated lysates were transferred to PVDF membranes (Transblot Turbo, Bio-Rad Labs, Hercules, CA). Membranes were probed for pS6, pERK and β-Actin (Cell Signaling Technology, Danvers, MA), signal detected on Chemidoc MP (Bio-Rad Labs, Hercules, CA), and quantified using ImageJ software.

### Inhibitor Establishment and Analysis of Plasma Samples

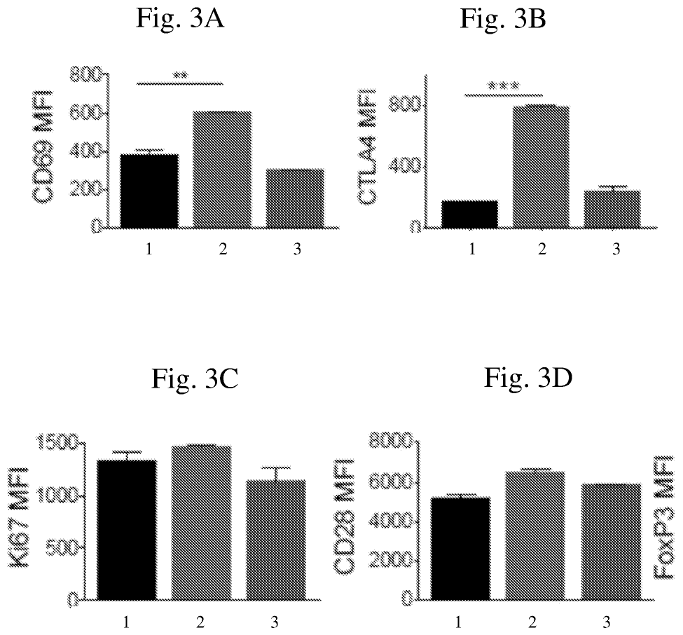

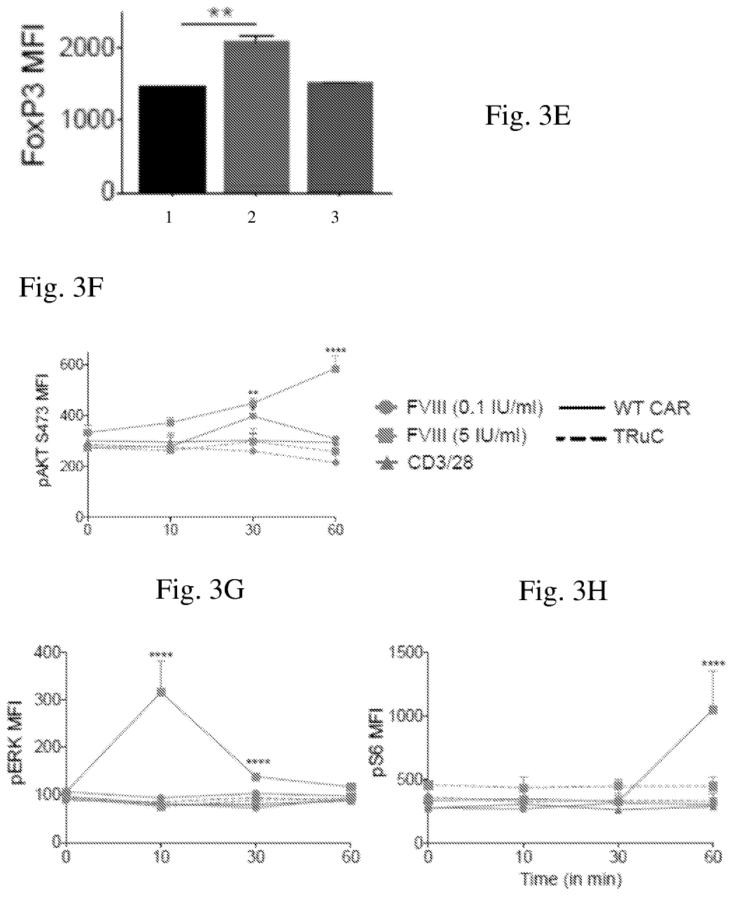

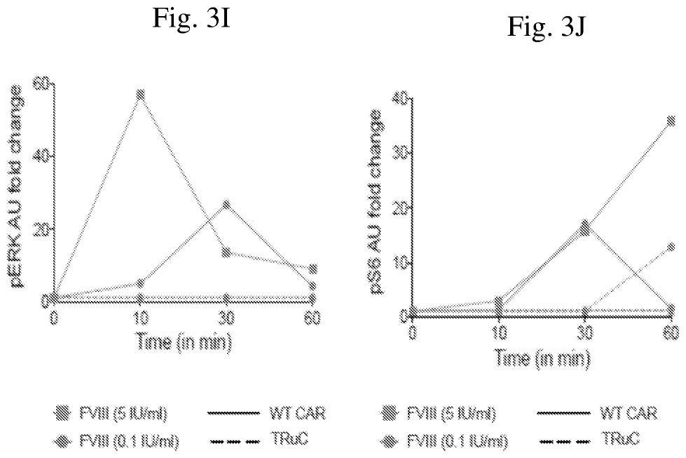

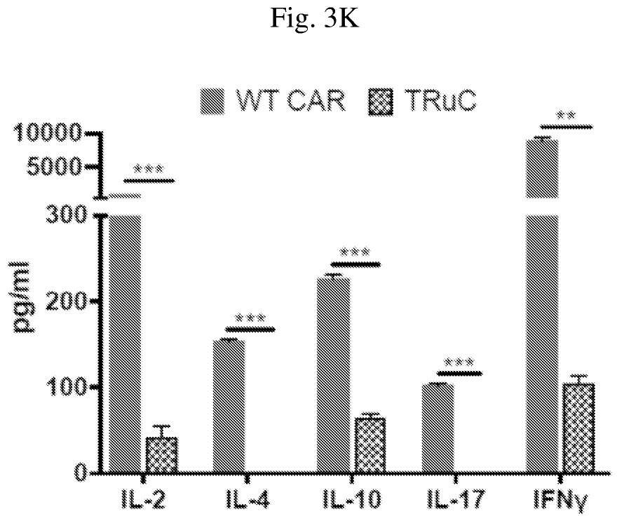

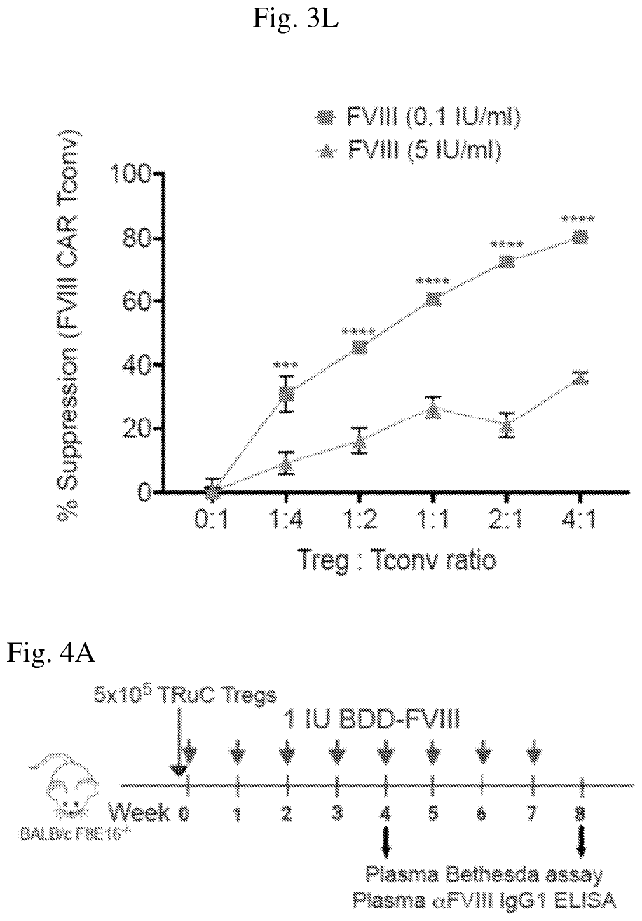

BALB/c F8e16^{-/-} hemophilia A (HA) mice (n=5-10) received weekly IV administrations of 1.5 IU BDD-FVIII. Mice received 5×10⁵ FVIII CAR or TRUC Tregs 1 day prior to starting BDD-FVIII injections (FIGS. 3F-3J). At 1- and 2-month time points post-adoptive transfer, ˜200 μl blood was collected from the retroorbital plexus using non-treated capillary tubes into 3.8% sodium citrate, and plasma was analyzed for inhibitor formation by the Bethesda assay (measured on a Diagnostica Stago STart Hemostasis Analyzer, Parsippany, NJ) and anti-FVIII IgG1 ELISA as previously described (Cao, et al. (2009) Mol Ther 17: 1733-1742). 1 Bethesda unit (BU) is defined as the reciprocal of the dilution of test plasma at which 50% of FVIII activity is inhibited.

### Statistical Analysis

Data shown are mean±SEM. Statistical significance was determined using the student's t-test, 1-way or 2-way ANOVA and multiple comparisons were made using Dunnett's, Tukey's, Sidak's or Kruskal-Wallis post-tests as indicated, using GraphPad Prism 8 software (La Jolla, CA). Values at P<0.05 were deemed significant and indicated as follows: *P<0.05, **P<0.01, ***P<0.001, ****P<0.0001. Frequencies of mice that developed inhibitors were compared using Fisher's exact test.

### Results

### Generation of a FVIII Directed CAR for Engineered Specificity.

We synthesized a second generation FVIII CAR construct comprising a high-affinity (10⁻¹¹M⁻¹) extracellular human scFv (BO2C11, which was specific for the highly immunogenic C2 domain of FVIII), complexed to the transmembrane and intracellular murine CD28 costimulatory and CD3ζ signaling domains. Tregs from BALB/c Foxp3^{IRES-GFP} mice, which express GFP under control of the mouse Foxp3 promoter, were magnetically enriched and purified by FACs sorting (>98% FoxP3^{GFP+} cells). Following transduction of activated Tregs with the FVIII CAR-pMYs-IRES-mScarlet retroviral vector, mScarlet and FoxP3^{GFP+} co-expressing Tregs were FACs sorted for a 2^{nd} time (>98% mScarlet⁺FoxP3^{GFP+}) and ex vivo expanded for a short period (3-4-days) to obtain ˜2-fold expansion. This was done in order to minimize phenotypic or functional differences that may arise as a result of prolonged ex vivo culture.

Detection of the c-Myc epitope tag on transduced Tregs confirmed surface scFv expression. Binding of Fc conjugated B domain deleted (BDD)-FVIII (FVIIIFc) was highly sensitive. Transduced murine FVIII CAR Tregs upregulated Treg associated activation markers, CD69, LAP, GITR, FoxP3, Ki67 and surface CD28 upon stimulation with BDD-FVIII or FVIIIFc for 48 h. In vitro stimulated CellTrace Violet (CTV) labeled CAR Tregs proliferated in an antigen specific manner (Division index 3.20±0.007, although proliferation was limited in the absence of exogenous IL-2 in the culture media. Lack of activation/proliferation in unstimulated cells or upon stimulation with an irrelevant antigen, FIXFc, suggested that tonic signaling in the absence of cognate ligand did not occur.

### ITAM Mutations in FVIII CAR Tregs Increase Persistence.

In vitro stimulation of FVIII CAR Tconv cells with either BDD-FVIII or FVIIIFc resulted in a significant loss of viability. We hypothesized that the additive signaling effect contributed by all three pairs of CD33 ITAMS might be responsible for activation induced cell death (AICD) in these transduced cells. We therefore mutated either the proximal (ITAM1⁻) or distal (ITAM3⁻) tyrosine residues in CD33. Mutating either ITAM1⁻ or ITAM3⁻ in order to disrupt the extent of CD3ζ signaling significantly prevented AICD in BDD-FVIII stimulated FVIII CAR Tconv. On the other hand, mutating both the proximal and distal residues (ITAM1⁻3⁻) completely abrogated CAR signaling and had no effect on viability. Interestingly, FVIII stimulated WT CAR Tregs did not develop AICD or any associated cytotoxicity, indicating comparative resistance to apoptosis. Single ITAM mutations did not markedly impede CAR Treg function, as expression of activation markers, CD69 and Ki67, did not differ significantly between FVIII stimulated WT, ITAM1⁻ or ITAM3⁻ mutated CAR Tregs. Following adoptive transfer and subsequent BDD-FVIII administration, all three CAR Treg variants had similar division indices in vivo (WT 7.5±0.8, ITAM1⁻ 7.3±0.4, ITAM3⁻ 8.1±2.2). ITAM1⁻ CAR Tregs exhibited increased persistence in spleens of recipient BALB/c F8e16^{-/-} mice as compared to WT CAR Tregs (0.30±0.1% vs 0.12±0.09%/splenic CD4+ T cells, day 3 post adoptive transfer). We incorporated the ITAM1⁻ mutation to all further modifications of the FVIII CAR construct.

The suppressive capacity of WT, ITAM1⁻, ITAM3⁻FVIII CAR Tregs was then assessed in vivo. Naïve BALB/c F8e16^{-/-} recipient mice were infused with 5×10⁵ sorted FVIII CAR Tregs, followed by 4-weekly intravenous (IV) injections of 1.5 IU BDD-FVIII. To our surprise, adoptively transferred FVIII CAR Tregs were found to be immune stimulatory, escalating the formation of inhibitors in recipient animals. Mice that received CAR Treg cellular therapy developed high titer inhibitors (55.5±6.4 BU/mL), as compared to controls that only received FVIII injections (7.3±1.0 BU/mL). In contrast, freshly isolated polyclonal thymic Tregs (1×10⁶ tTregs) were suppressive (2.4±1.7 BU/mL). αFVIII IgG1 levels corroborated these findings. Ex vivo expansion of CAR Tregs in the presence of rapamycin did not restore suppressive function in FVIII CAR Tregs, although it was able to prevent inhibitor escalation to some extent (14.9±5.4 BU/mL), indicating that signaling pathways downstream of CD3ζ such as mTOR might regulate CAR signaling effects.

### Dose Dependent Dysregulation of Signaling.

We evaluated cytokine secretion and transcription factor co-expression by BDD-FVIII stimulated CAR Tregs. Activated WT CAR Tregs produced high levels of IL-10, IL-4, IFNγ, comparable to FVIII CAR Tconv cells, and low levels of IL-2 and IL-17 at 48 h. Intracellular staining confirmed a heterogenous cytokine profile in the transduced Treg population, with predominant expression of IFNγ in the FoxP3⁺ population. This heterogenous profile was also observed at the transcription factor level with upregulation of IRF4, T-bet, and GATA3, typically associated with either Th1 or Th2 responses, as quantified by both flow cytometry and real time RT-PCR.

We sought to understand the basis for this dysregulation by first looking at the effect of antigen dose. We speculate that tTregs might exhibit a high affinity for self-antigen at antigen doses that might be too low for Tconv stimulation. Whether high antigen dose can, in turn, destabilize Treg suppressive function is not known. We performed an in vitro suppression assay to determine if FVIII CAR Tregs could suppress the proliferation of FVIII CAR Tconv responders when stimulated with high dose (5 IU/mL) or low dose (0.1 IU/mL) BDD-FVIII. Low dose BDD-FVIII stimulated FVIII CAR Tregs were able to suppress the proliferation of FVIII CAR Tconv cells even at low Treg:Tconv ratios. This suppressive effect was lost on stimulation with high dose BDD-FVIII. Non-specific suppression was observed at a high Treg:Tconv ratio, which could be attributed to competition for antigen and IL-2. We then performed phospho-flow analysis of signaling molecules downstream of TCR/CD3ζ, which are amplified by CD28 engagement, such as the PI3K-PDK1-AKT and the MAPK/ERK pathways. We observed greatly enhanced phosphorylation of AKT (S473) and S6 kinases at 30-60 min, with a rapid transient response time for ERK at 10 min in high dose BDD-FVIII stimulated FVIII CAR Tregs. In contrast, dampened phosphorylation of AKT (T308), AKT (S473), S6 and ERK was seen in transduced Tregs on TCR triggering (anti-CD3/28 microbeads) or low dose BDD-FVIII CAR stimulation. FVIII CAR Tconv cells triggered with CD3/28 microbeads were used as a positive control for S6, ERK and AKT (S473) phosphorylation, which confirmed that TCR signaling in Tconv cells was much more robust as compared to Tregs.

### Mutations in CD28 Signaling Motifs Impact FVIII CAR Treg Cytokine Profile.

Cytokine signaling is responsive to signals emanating from both the TCR and the costimulatory receptor. Since CD28 is known to increase the rate of CD35 phosphorylation, potentiate TCR signaling and increase effector cytokine production, we generated targeted mutations in the CD28 signaling motifs, YMNM or PYAP, known to bind PI3K and LCK kinases respectively. CD28-Y170F or CD28-AYAA substitution mutations did not negatively affect the upregulation of activation markers CD69, CD28, Ki67 or CTLA-4 in response to BDD-FVIII stimulation in vitro. Notably, both the CD28-Y170F and CD28-AYAA mutations significantly reduced production of IFNγ and IL-4 in BDD-FVIII stimulated CAR Tregs, although this was also accompanied by diminished IL-10 production. In vivo, however, CD28-Y170F and CD28-AYAA FVIII CAR Tregs were still unable to suppress inhibitor formation (4.25±2.15, 9.1±1.0, 2.87±1.58 BU/mL for control, CD28-Y170F and CD28-AYAA groups respectively), although we did not observe high inhibitor escalation.

Constitutive IL-10 Expression does not Restore CAR Treg Function.

As a second strategy, we incorporated the murine IL-10 coding sequence downstream of the auto-cleaving P2A peptide sequence in WT, CD28-Y170F or CD28-AYAA knock-in variants of FVIII CAR. IL-10 is an important modulator in Tregs that is known to regulate the production of both Th1 and Th2 cytokines. IL-10 coding CARs constitutively produced IL-10 (1617-2260 pg/mL), which increased 1.3 to 2.1-fold on in vitro BDD-FVIII stimulation. IFNγ, IL-2 IL-17, IL-4 and TNFα levels were either completely abrogated or significantly diminished in IL-10 overexpressing WT, CD28-Y170F or CD28-AYAA CAR Tregs. Notably, IL-10 overexpression did not affect the ability of the CAR Tregs to proliferate in response to BDD-FVIII stimulation in vitro. However, constitutive overexpression of IL-10 in adoptively transferred FVIII CAR Tregs or combined with targeted mutations in CD28 was unable to tolerize recipient BALB/c F8e16^{-/-} mice (4.5±1.5, 41.3±9.2, 36±12.3, 29.1±8.17 BU/mL for control, WT-IL-10, CD28-AYAA-IL10 and CD28-Y170F-IL-10 cohorts respectively).

Integration of TRUC into the TCR-CD3 Complex Regulates Surface Expression.

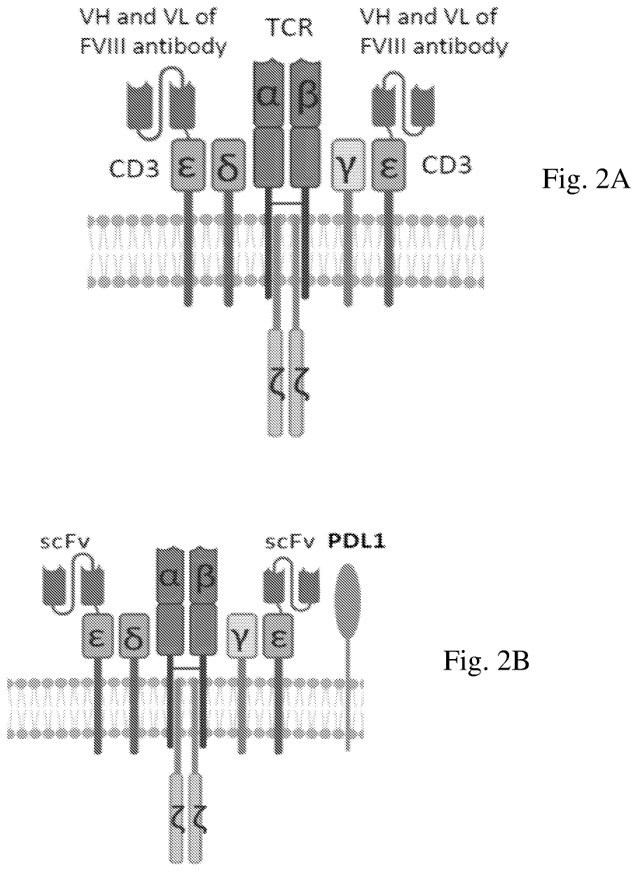

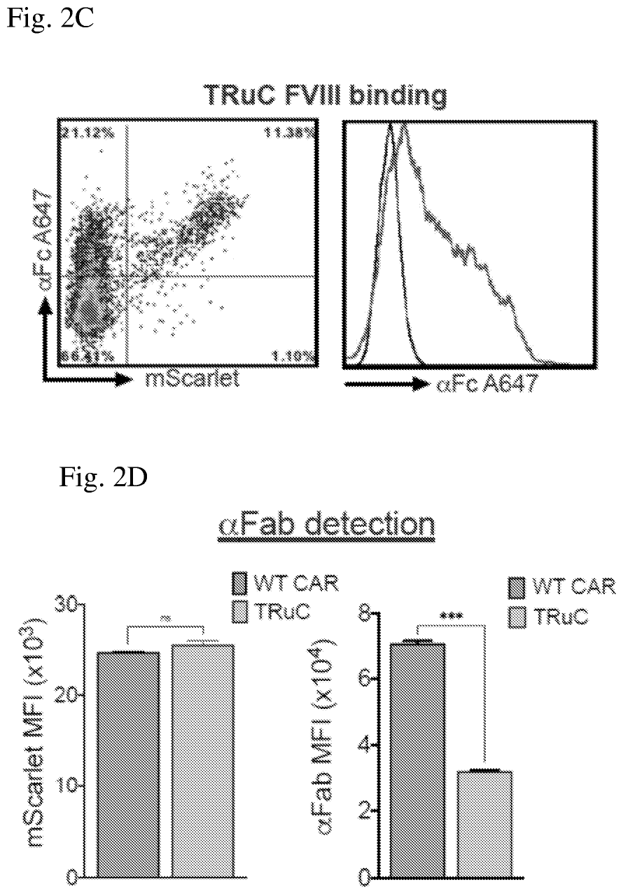

A recent report showed that fusing anti-CD19 scFv to the N-termini of any of the five TCR subunits results in incorporation of TRuCs into the TCR-CD3 complex. This approach significantly improved tumor cell lysis as compared to high-affinity CD19-CAR T cells, which correlated with differences in intracellular signaling events between the two constructs. We fused FVIII scFv to murine CD3ε in order to generate FVIII TRUC Tregs (FIG. 2A). FVIII TRUC was surface expressed in transduced Tregs and bound FVIIIFc in vitro. However, we observed under conditions of comparable reporter protein expression (mScarlet MFI) that surface scFv expression, detected by Fab antibody, was markedly lower for FVIII TRUC as compared to FVIII CAR. This indicates that TRUC surface expression is limited by the number of TCR-CD3 complexes on the transduced Treg. In order to confirm FVIII TRUC integration into the TCR-CD3 complex we co-transduced the murine T cell hybridoma 5KCα⁻/β⁻, which are TCRα/β deficient, with FVIII TRUC or FVIII CAR and a murine TCRα/β expressing construct. We observed that FVIII CAR was surface expressed, bound FVIIIFc and upregulated the activation marker CD69 independent of TCRα/β expression. In contrast, FVIIIFc binding and CD69 upregulation in FVIII TRUC expressing 5KCα⁻/β⁻ cells was dependent on co-transduction with TCRα/β, which confirms that TRUC integrates into the TCR-CD3 complex (FIG. 2D). Next, we tested the requirement for both TCRα/β and CD3 for TRUC incorporation. HEK-293 cells were transfected with FVIII CAR or TRUC and either a murine TCRα/β or CD3δγεζ expressing construct, or both. Whereas FVIIIFc binding by FVIII CAR was independent of TCRα/β or the CD3 complex, FVIII TRUC surface expression and FVIIIFc binding depended on co-transfection of both TCRα/β and CD3.

### TRUC Tregs Exhibit Controlled Signaling.

BDD-FVIII stimulation of FVIII TRUC Tregs in vitro led to upregulation of CD69, Ki67, CD28, FoxP3, and a 5-fold increase in CTLA4 expression (FIGS. 3A-3E). In contrast to FVIII CAR Treg, phosphorylation of signaling molecules AKT (S473), ERK and S6 was considerably dampened in TRUC Tregs, similar to levels observed on TCR triggering with anti-CD3/28 microbeads (FIG. 3B). This was also confirmed by western blot for pERK and pS6 (FIGS. 3F-3J). FVIII TRUC Tconv cells triggered with CD3/28 microbeads were used as a positive control for S6, ERK and AKT (S473) phosphorylation, which confirmed that Tconv cell signaling was much more robust as compared to Tregs.

Consistent with dampened signaling, BDD-FVIII stimulated TRUC Tregs secreted significantly lower levels of cytokines IL-2, IL-4, IL-17, IL-10 and IFNγ as compared to WT CAR Tregs, (FIG. 3K). This was also confirmed by intracellular cytokine staining, which showed low but significantly elevated frequencies of IL-10 in BDD-FVIII stimulated TRUC Tregs and insignificant IFNγ expression. Similar to FVIII CAR Tregs, FVIII TRUC Tregs were able to suppress the in vitro proliferation of TRuC Tconv responders when stimulated with low dose (0.1 IU/mL) BDD-FVIII (FIG. 3L). This suppressive effect was lost on stimulation with high dose BDD-FVIII. We also tested to see if engineered Tregs induce cytolysis by evaluating granzyme B and CD107a upregulation. Neither CAR or TRUC Tregs upregulated cytolytic markers in response to BDD-FVIII or CD3/28 stimulation, whereas CAR and TRUC Tconv cells exhibited substantial increase in both granzyme B and CD107a expression on CD3/28 stimulation, and a less significant increase on BDD-FVIII stimulation.

TRUC Tregs are Suppressive In Vivo but have Limited Persistence.

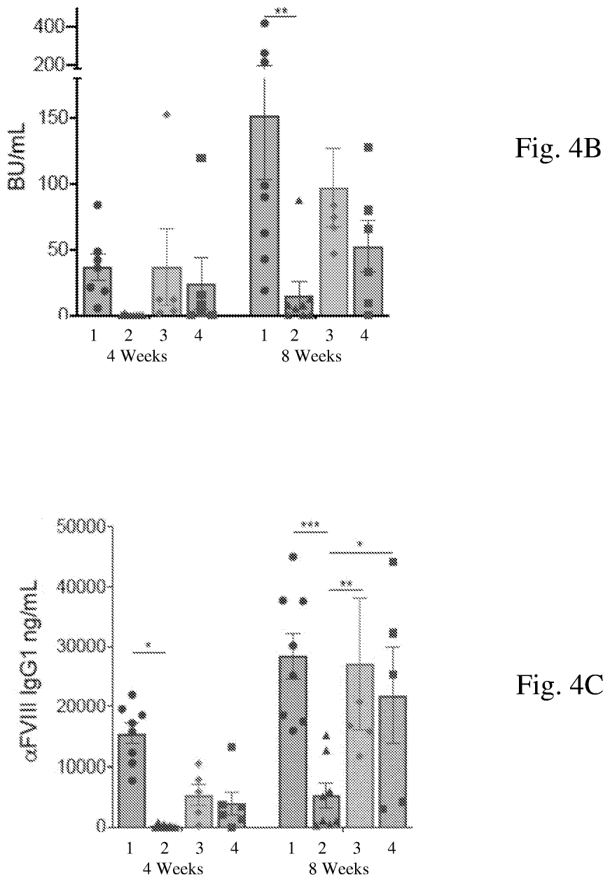

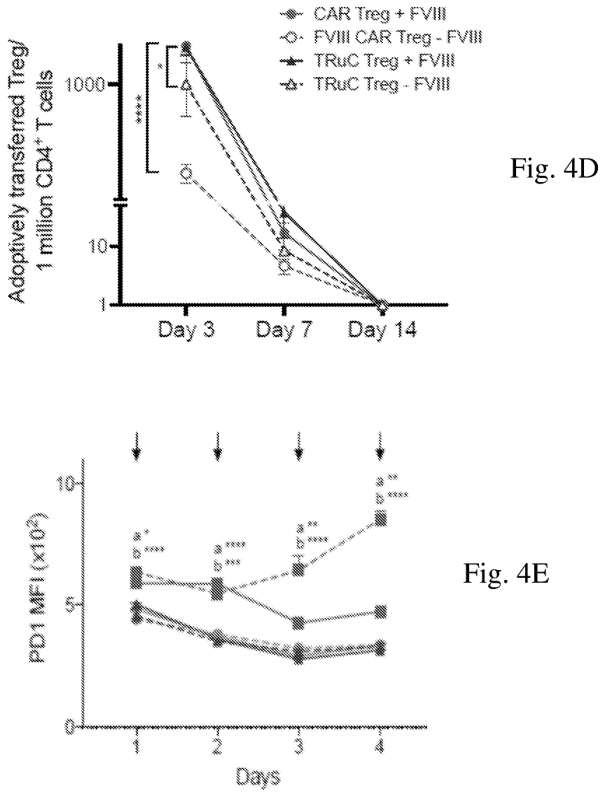

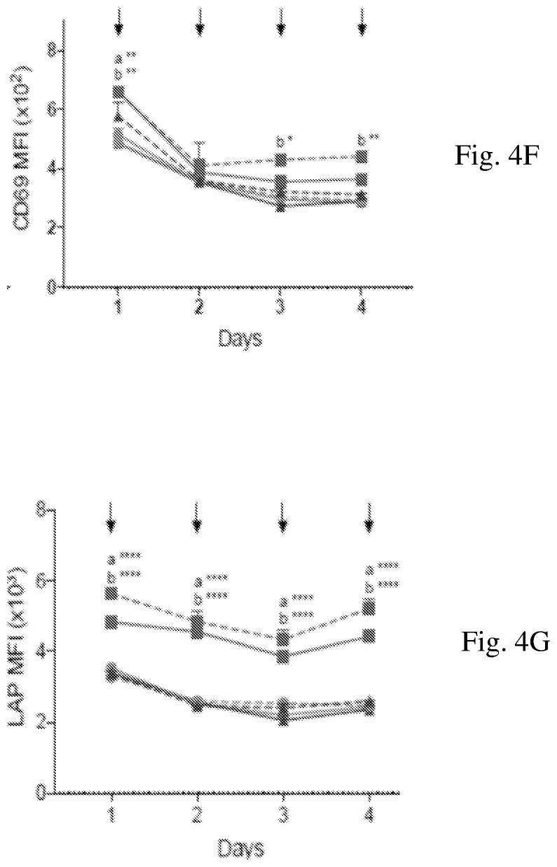

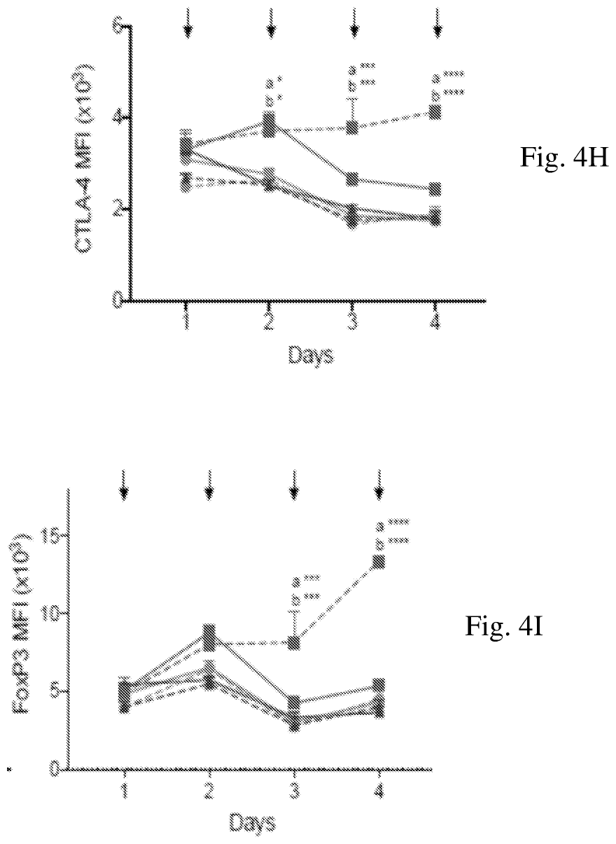

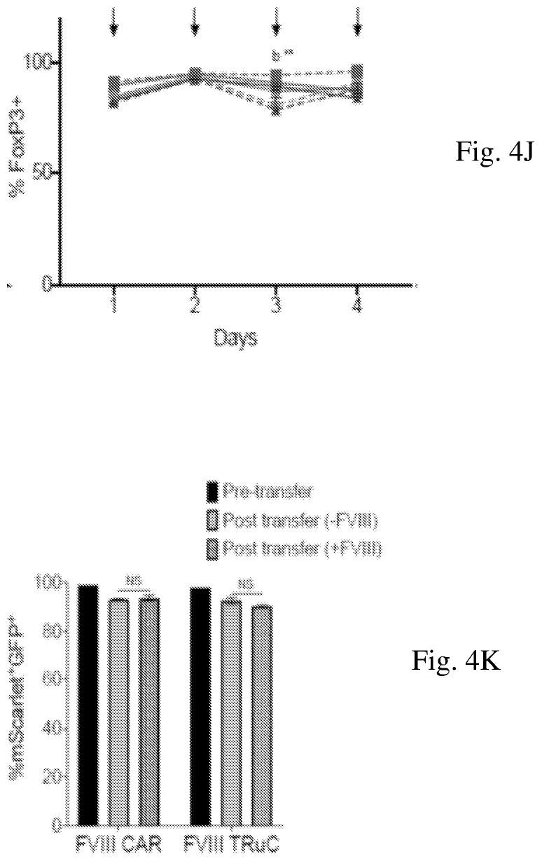

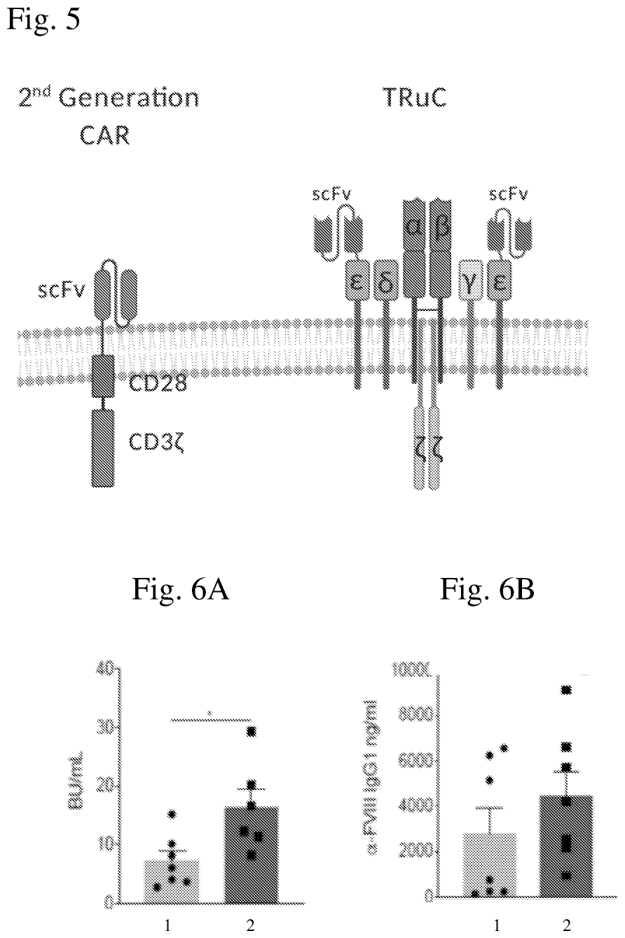

We investigated whether controlled signaling by TRUC Tregs was sufficient to maintain a suppressive phenotype in vitro and in vivo. In vivo, naïve BALB/c F8e16^{-/-} recipient mice were infused with 5×10⁵ sorted TRUC Tregs or polyclonal tTregs (5×10⁵ or 1×10⁶) followed by 8 weekly IV injections of 1.5 IU BDD-FVIII (FIG. 4A). FVIII TRUC Tregs were more effective at suppressing inhibitor formation as compared to polyclonal tTregs (FIG. 4B, FIG. 4C). 7 out of 8 animals in the TRUC Treg group did not develop detectable inhibitors (avg BU/mL 0.23±0.23) at 4 weeks, whereas 100% of mice in the control group developed high titer inhibitors >5 BU/mL (avg BU/mL 36.48±9.66, p=0.02). At 8 weeks, mice in the FVIII TRUC Treg group had a mean inhibitor titer of 15.4±10.4 BU/mL as compared to the control group (avg BU/mL 151.4±48.6, p=0.004, FIG. 4B). αFVIII IgG1 titers were also significantly lower in the FVIII TRUC Treg group (5238±3862 ng/mL) as compared to the polyclonal tTreg groups at 8 weeks (5×10⁵ tTreg group: 28429±3862 ng/mL, p=0.004, 1×10⁶ tTreg group: 21821±8020 ng/mL, p=0.04), suggesting a more sustained tolerogenic effect for FVIII TRUC Tregs (FIG. 4C). Although adoptively transferred Tregs persisted only transiently (7-14 days), we observed that BDD-FVIII stimulation was required for increased persistence in vivo. Repeated BDD-FVIII injections led to 1.75-fold (p=0.0001) and 1.17-fold (p=0.036) higher numbers of CAR Tregs and TRUC Tregs respectively on Day 3 (FIG. 4D).

### BDD-FVIII Stimulated CAR and TRUC Tregs Maintain a Treg Phenotype.

We next asked whether BDD-FVIII stimulation would affect CAR and TRUC Treg stability. To address this, we performed repeated (daily) stimulations of CAR Treg and TRUC Treg with BDD-FVIII in vitro and evaluated their phenotype after each stimulation. FoxP3 frequencies were unchanged in both BDD-FVIII stimulated CAR and TRUC Tregs (FIG. 4E-4J). PD1, CD69, LAP, CD69 and FoxP3 MFI were significantly elevated in BDD-FVIII stimulated CAR and TRUC Tregs, as compared to unstimulated controls or FIX stimulated controls (FIG. 4E).

Finally, we analyzed the phenotype of sorted mScarlet⁺FoxP3^{GFP+} cells prior to and post adoptive transfer into BALB/c F8e16^{-/-} mice. Pre-adoptive transfer, mScarlet⁺FoxP3^{GFP+} cells were 92-95% FoxP3⁺ with a good correlation between FoxP3 and GFP (FIG. 4K). Cohorts of recipient mice were either left untreated or injected with BDD-FVIII every 2 days, starting 1 day post adoptive transfer. On day 3 post adoptive transfer, splenic CD4⁺ T cells were magnetically enriched and frequencies of mScarlet⁺FoxP3^{GFP+} cells were evaluated. mScarlet⁺FoxP3^{GFP+} CAR Treg and TRUC Treg both retained GFP expression, which correlated with FoxP3 expression (CAR Treg 93.18±0.36%, TRUC Treg 92.75±0.3% mScarlet⁺FoxP3^{GFP+} cells). Treg phenotype was not affected by BDD-FVIII stimulation in vivo (CAR Treg 93.71±0.7%, TRUC Treg 90.54±0.3% mScarlet⁺FoxP3^{GFP+} cells) (FIG. 4K). Overall, these results demonstrate that CAR and TRUC mediated signaling preserves the regulatory phenotype of engineered Tregs.

### Example 2

### Enhancing Long Term Suppression of ADA

Localization to germinal centers. We will first initiate a GC CXCL13 gradient by administering 3-4 weekly FVIII IV injections in HA mice (n=6/group). 2×106 CellTrace Violet labeled FVIII TRUC, FVIII TRUC CXCR5, FVIII TRUC PD-L1 or FVIII TRUC CXCR5+PD-L1 Tregs will be adoptively transferred and immediately followed up with an additional FVIII injection. 72 h after adoptive transfer, spleens will be perfused/fixed, equilibrated in sucrose, mounted, snap frozen and stored at −80° C. 7-8 μm cryosections will be immunofluorescence stained for Tfh and Tfr cells (CD4+, CXCR5+, FoxP3+), follicular dendritic cells (CD11c+CD35+). Number and average size of GCs (GL7hiIgDlo clusters located within IgD+follicles) in adoptive transfer recipient or control mice will be compared (Fiji software). We will stain with Ki67 for rapid identification of GC structures within B cell follicles. We will quantify distribution of adoptively transferred Tregs in the T cell zone, T-B border, B cell follicles and GCs.

Effect on Tfh and GC cells. HA mice (n=12/group) will be adoptively transferred with 0.5×106 FVIII TRUC, FVIII TRUC CXCR5, FVIII TRUC PD-L1, FVIII TRUC CXCR5+PD-L1 Tregs or mock transfer, followed 24 h later by weekly 1.5IU FVIII injections, as in FIGS. 4A-4K. Mice will be euthanized at 4 weeks, during which we have earlier identified peak Tfh and GC responses. We will evaluate Tfh and Tfr (CD4+PD1+ICOS+CXCR5+FoxP3−/+Bc16+), memory B cell (CD19+CD138-GL7-IgD-IgM/G+CD38+), GC (CD19+GL7+CD95+), plasmablast (CD19+CD138+TACI+) and short-lived plasma cell (CD19-CD138+TACI+) populations from spleens of adoptive transfer recipients and mock transfer mice. We will quantify frequencies, total numbers, Tfh:Tfr ratios, activation markers CD44, CD69, Ki67, CD40L166, intracellular staining for IL-21 on Tfh cells and co-expression of IL-4, IFNγ, or IL-10 by flow cytometry. We will test plasma levels of IL-2, IL-4, IFNγ, IL-10, TGF-b cytokines by ELISA.

ADA suppression. HA mice (n=12/group) will be adoptively transferred with modified and unmodified FVIII TRUC Tregs, followed by weekly IV injections with FVIII as outlined in FIGS. 4A-4K. Functional ADAs will be quantified from plasma by Bethesda assay and IgM/IgG1/IgG2a ELISA. We will test for risk of off-target suppressive effects by administering the unrelated antigen keyhole limpet hemocyanin (KLH, 100 μg intraperitoneally) 1 month following engineered Treg administration. Both mice that received KLH alone or engineered Treg therapy+ KLH should develop robust Ab responses to the T dependent KLH antigen.

In conclusion, we propose that the TRUC design provides superior suppressive capacity to Tregs, and that this suppressive capacity is maintained over a range of scFv affinities.

The feasibility of our strategies to engineer improved stability, suppression and persistence in TRUC Tregs is supported by our preliminary studies and will generate a superior protocol for cellular therapy. The present disclosure supports a generalized design that is applicable to most antibody-based receptors using a TCR signaling platform which will facilitate translation of engineered Treg therapy for various clinical indications.

1. A method of modifying an inappropriate immune response in a patient receiving repeated doses of a therapeutic exogenous agent, said method comprising
administering modified Tregs to said patient to suppress the formation of antibodies against the therapeutic agent, wherein said modified Tregs comprise a T cell receptor fusion construct (TRUC), said TRUC comprising
variable heavy (VH) and light (VL) chains of an antibody with specificity to said therapeutic agent protein; and
a T cell receptor CD3 epsilon subunit, wherein said variable heavy (VH) and light (VL) chains are fused to the CD3 epsilon subunit optionally via a peptide linker.

2. The method of claim 1 wherein said TRUC comprises a single chain variable fragment (scFv) having specificity for said therapeutic agent fused to the N-terminus of a CD3ε subunit, optionally via a peptide linker.

3. (canceled)

4. The method of claim 3 wherein the administered Tregs are autologous that have been modified to comprises said TRUC.

5. The method of claim 2 wherein the therapeutic agent is clotting factor FVIII.

6. The method of claim 1 wherein the TRUC further comprises hinge regions selected from either murine or human CD28 or CD8.

7. The method of claim 1 wherein said Treg further expresses a gene encoding chemokine receptor CXCR5.

8. (canceled)

9. The method of 4 wherein said TRUC comprises an amino acid sequence having at least 95% sequence identity to SEQ ID NO: 4.

10.-15. (canceled)

16. An engineered Treg, where the endogenous TCR-CD3 signaling is reconfigured to respond to scFv-based recognition, said Treg expressing
i) a TCR fusion construct comprising
a single chain variable fragment (scFv) having specificity for a preselected therapeutic agent, said scFv being fused to the N-terminus of a CD3ε subunit, optionally via a peptide linker.

17. The engineered Treg of claim 16 wherein the Treg further expresses an additional gene, Programmed Death Ligand 1 (PDL1).

18. The engineered Treg of claim 16 wherein said Treg further expresses a gene encoding chemokine receptor CXCR5.

19. The engineered Treg of claim 18 wherein said scFv has specificity for clotting factor FVIII.

20. The engineered Treg of claim 19 wherein said scFV specifically binds to amino acid residues 2125-2332 of human FVIII.

21. The engineered Treg of claim 16 wherein the TCR fusion construct further comprises hinge regions selected from either murine or human CD28 or CD8.

22. The engineered Treg of claim 16 wherein said TCR fusion construct comprises an amino acid sequence having at least 95% sequence identity to SEQ ID NO: 4.

23. The engineered Treg of claim 16 wherein said TCR fusion construct comprises an amino acid sequence corresponding to SEQ ID NO: 4, wherein the murine amino acid sequences have been substituted with the corresponding human equivalent amino acid sequences.

24. A TCR fusion construct comprising
single chain variable fragment (scFv) having specificity for clotting factor FVIII fused to the N-terminus of a CD3ε subunit, optionally via a peptide linker.

25. The TCR fusion construct of claim 24 comprising an amino acid sequence having at least 95% sequence identity to a sequence selected from SEQ ID NO: 2 or 4.

26. The TCR fusion construct of claim 24, wherein a nucleic acid construct comprises a polynucleotide encoding the TCR fusion construct comprising single chain variable fragment (scFv) having specificity for clotting factor FVIII fused to the N-terminus of a CD3ε subunit, optionally via a peptide linker.

27. The nucleic acid construct of claim 26 further comprising a nucleic acid sequence encoding Programmed Death Ligand 1 (PDL1).

28. The nucleic acid construct of claim 26 wherein said nucleic acid has at least 95% sequence identity to a sequence selected from SEQ ID NO: 1 or 3.


SEQUENCE LISTING (6 sequences)

SEQ ID NO: 1 - DNA, 1338 bp, synthetic construct
  misc_feature 1..1338: note = chimeric antigen receptor
  source 1..1338: mol_type = other DNA
atgcggtggaacactttctggggcatcctgtgcctcagcctcctagctgttggcacttgccaggacgaaatcgccctcactcagagcccaggcaccctgagcctgtcccctggagagagggccacactgtcttgcagagcttctcagagcttcagctcctcttacctggcctggtaccagcagaagcctggccaggctccaaggctgctgatctacggagctagcacccgggctacaggaatcccagaccgcttctccggatctggcagcggaaccgacttcaccctgacaatcagcaggctggagcccgaggacttcgccgtgtactactgccagaagtacggcacctccgctatcacatttggccagggaacaagactggagatcaagggaggaggaggaggatctggaggaggaggctctggaggaggaggctcccaggtgcagctggtgcagagcggagctgaggtgaagaagcctggagcttctgtgaaggtgagctgtaaggtgtccggctacaccctgacagagctgccagtgcactgggtgcggcaggctccaggcaagggactggagtgggtgggctccttcgatccagagagcggagagtccatctacgctcgcgagtttcagggctctgtgaccatgacagccgacaccagcacagatatcgcttacatggagctgagctccctgagatctgacgataccgccgtgtactactgcgctgtgcccgaccctgatgccttcgacatctggggccagggaaccatggtgacagtgtctagcggaggaggaggatctggaggaggaggctctggaggaggaggctccgatgccgagaacattgaatacaaagtctccatctcaggaaccagtgtagagttgacgtgccctctagacagtgacgagaacttaaaatgggaaaaaaatggccaagagctgcctcagaagcatgataagcacctggtgctccaggatttctcggaagtcgaggacagtggctactacgtctgctacacaccagcctcaaataaaaacacgtacttgtacctgaaagctcgagtgtgtgagtactgtgtggaggtggacctgacagcagtagccataatcatcattgttgacatctgtatcactctgggcttgctgatggtcatttattactggagcaagaataggaaggccaaggccaagcctgtgacccgaggaaccggtgctggtagcaggcccagagggcaaaacaaggagcggccaccacctgttcccaacccagactatgagcccatccgcaaaggccagcgggacctgtattctggcctgaatcagagagcagtctga

SEQ ID NO: 2 - protein, 445 aa, synthetic construct
  REGION 1..445: note = chimeric antigen receptor
MRWNTFWGILCLSLLAVGTCQDEIALTQSPGTLSLSPGERATLSCRASQSFSSSYLAWYQQKPGQAPRLLIYGASTRATGIPDRFSGSGSGTDFTLTISRLEPEDFAVYYCQKYGTSAITFGQGTRLEIKGGGGGSGGGGSGGGGSQVQLVQSGAEVKKPGASVKVSCKVSGYTLTELPVHWVRQAPGKGLEWVGSFDPESGESIYAREFQGSVTMTADTSTDIAYMELSSLRSDDTAVYYCAVPDPDAFDIWGQGTMVTVSSGGGGSGGGGSGGGGSDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAV

SEQ ID NO: 3 - DNA, 2274 bp, synthetic construct
  misc_feature 1..2274: note = chimeric antigen receptor
  source 1..2274: mol_type = other DNA
atgcggtggaacactttctggggcatcctgtgcctcagcctcctagctgttggcacttgccaggacgaaatcgccctcactcagagcccaggcaccctgagcctgtcccctggagagagggccacactgtcttgcagagcttctcagagcttcagctcctcttacctggcctggtaccagcagaagcctggccaggctccaaggctgctgatctacggagctagcacccgggctacaggaatcccagaccgcttctccggatctggcagcggaaccgacttcaccctgacaatcagcaggctggagcccgaggacttcgccgtgtactactgccagaagtacggcacctccgctatcacatttggccagggaacaagactggagatcaagggaggaggaggaggatctggaggaggaggctctggaggaggaggctcccaggtgcagctggtgcagagcggagctgaggtgaagaagcctggagcttctgtgaaggtgagctgtaaggtgtccggctacaccctgacagagctgccagtgcactgggtgcggcaggctccaggcaagggactggagtgggtgggctccttcgatccagagagcggagagtccatctacgctcgcgagtttcagggctctgtgaccatgacagccgacaccagcacagatatcgcttacatggagctgagctccctgagatctgacgataccgccgtgtactactgcgctgtgcccgaccctgatgccttcgacatctggggccagggaaccatggtgacagtgtctagcggaggaggaggatctggaggaggaggctctggaggaggaggctccgatgccgagaacattgaatacaaagtctccatctcaggaaccagtgtagagttgacgtgccctctagacagtgacgagaacttaaaatgggaaaaaaatggccaagagctgcctcagaagcatgataagcacctggtgctccaggatttctcggaagtcgaggacagtggctactacgtctgctacacaccagcctcaaataaaaacacgtacttgtacctgaaagctcgagtgtgtgagtactgtgtggaggtggacctgacagcagtagccataatcatcattgttgacatctgtatcactctgggcttgctgatggtcatttattactggagcaagaataggaaggccaaggccaagcctgtgacccgaggaaccggtgctggtagcaggcccagagggcaaaacaaggagcggccaccacctgttcccaacccagactatgagcccatccgcaaaggccagcgggacctgtattctggcctgaatcagagagcagtctgaggaagcggagctactaacttcagcctgctgaagcaggctggagacgtggaggagaaccctggacctatgaggatatttgctggcattatattcacagcctgctgtcacttgctacgggcgtttactatcacggctccaaaggacttgtacgtggtggagtatggcagcaacgtcacgatggagtgcagattccctgtagaacgggagctggacctgcttgcgttagtggtgtactgggaaaaggaagatgagcaagtgattcagtttgtggcaggagaggaggaccttaagcctcagcacagcaacttcagggggagagcctcgctgccaaaggaccagcttttgaagggaaatgctgcccttcagatcacagacgtcaagctgcaggacgcaggcgtttactgctgcataatcagctacggtggtgcggactacaagcgaatcacgctgaaagtcaatgccccataccgcaaaatcaaccagagaatttccgtggatccagccacttctgagcatgaactaatatgtcaggccgagggttatccagaagctgaggtaatctggacaaacagtgaccaccaacccgtgagtgggaagagaagtgtcaccacttcccggacagaggggatgcttctcaatgtgaccagcagtctgagggtcaacgccacagcgaatgatgttttctactgtacgttttggagatcacagccagggcaaaaccacacagcggagctgatcatcccagaactgcctgcaacacatcctccacagaacaggactcactgggtgcttctgggatccatcctgttgttcctcattgtagtgtccacggtcctcctcttcttgagaaaacaagtgagaatgctagatgtggagaaatgtggcgttgaagatacaagctcaaaaaaccgaaatgatacacaattcgaggagacg

SEQ ID NO: 4 - protein, 757 aa, synthetic construct
  REGION 1..757: note = chimeric antigen receptor
MRWNTFWGILCLSLLAVGTCQDEIALTQSPGTLSLSPGERATLSCRASQSFSSSYLAWYQQKPGQAPRLLIYGASTRATGIPDRFSGSGSGTDFTLTISRLEPEDFAVYYCQKYGTSAITFGQGTRLEIKGGGGGSGGGGSGGGGSQVQLVQSGAEVKKPGASVKVSCKVSGYTLTELPVHWVRQAPGKGLEWVGSFDPESGESIYAREFQGSVTMTADTSTDIAYMELSSLRSDDTAVYYCAVPDPDAFDIWGQGTMVTVSSGGGGSGGGGSGGGGSDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAVGSGATNFSLLKQAGDVEENPGPMRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 5 - protein, 15 aa, synthetic construct
GGGGSGGGGSGGGGS

SEQ ID NO: 6 - protein, 167 aa, Mus musculus
DAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAV